SEQUENCE LISTING (10 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

(19) United States
(12) Patent Application Publication
(10) Pub. No.: US 2026/0139315 A1
(43) Pub. Date: May 21, 2026

(54) DIAGNOSTIC AND THERAPEUTIC METHODS FOR CANCER

(71) Applicants: Genentech, Inc., South San Francisco, CA (US); Hoffmann-La Roche Inc., Little Falls, NJ (US)

(72) Inventors: Rajat Mohindra, Reinach BL (CH); Ashis Saha, Burlingame, CA (US); G Scott Chandler, Basel (CH); Christian Hammer, Walnut Creek, CA (US)

(73) Assignees: Genentech, Inc., South San Francisco, CA (US); Hoffmann-La Roche Inc., Little Falls, NJ (US)

(21) Appl. No.: 19/453,797

(22) Filed: Jan. 20, 2026

Related U.S. Application Data
(63) Continuation of application No. PCT/US2024/038705, filed on Jul. 19, 2024.
(60) Provisional application No. 63/514,948, filed on Jul. 21, 2023.

(51) Int. Cl. C12Q 1/6883; C12Q 1/52; C12Q 1/6869

(52) U.S. Cl. CPC C12Q 1/6883; C12Q 1/52; C12Q 1/6869; C12Q 2600/106; C12Q 2600/156; G01N 2333/91188

(57) ABSTRACT

Provided herein are diagnostic and therapeutic methods for the treatment of cancer using polygenic risk scores (PRSs) for liver damage. In particular, the invention provides methods for patient selection and methods of treatment.

### CROSS REFERENCE TO RELATED APPLICATIONS

This application is a continuation of the International Application No. PCT/US202/038705, filed Jul. 19, 2024, which claims priority to U.S. Provisional Application No. 63/514,948, filed Jul. 21, 2023, each of which is herein incorporated by reference in its entirety.

### SEQUENCE LISTING

The present application contains a Sequence Listing which has been submitted in XML format and is hereby incorporated by reference in its entirety. Said XML copy, created on Jan. 20, 2026, is named P38505-US-1-Sequence-Listing.xml and is 10,556 bytes in size.

### FIELD OF THE INVENTION

Provided herein are diagnostic and therapeutic methods for the treatment of cancer using polygenic risk scores (PRSs) for liver damage and methods for identifying patients who are likely to develop liver dysfunction, e.g., hepatitis, under anti-cancer therapy. In particular, the invention provides methods for patient selection and methods of treatment.

### BACKGROUND

Cancer remains one of the most deadly threats to human health. In the U.S., cancer affects more than 1.7 million new patients each year and is the second leading cause of death after heart disease, accounting for approximately 1 in 4 deaths. It is also predicted that cancer may surpass cardiovascular diseases as the number one cause of death within 5 years.

Immune checkpoint inhibition has emerged as a promising treatment for some cancers, including cancers with high unmet need for treatment options. In healthy tissues, immune checkpoints function in the prevention of autoimmunity by limiting the activity of T-cells. Tumor cells may co-opt this mechanism to escape immune surveillance. Considerable attention has thus been given to therapies that suppress the function of immune checkpoints (e.g., immune checkpoint blockade) in patients having a cancer. A particular immune checkpoint protein of interest is programmed cell death protein-1 (PD-1 or CD279), which acts to limit the activity of T-cells in peripheral tissues. Blockade of PD-1 by a monoclonal antibody specific for PD-1 or its ligands, programmed death-ligand 1 (PD-L1; CD274) and programmed death-ligand 2 (PD-L2; CD273), has been shown to elicit durable anti-tumor responses in a subset of patients undergoing treatment for cancer.

Immune checkpoint inhibition has been associated with on-target toxicities that are referred to as immune-related adverse events (irAEs). However, irAEs occur only after immune checkpoint blockade therapy has been initiated.

Thus, there exists an unmet need for diagnostic and therapeutic approaches that enable the use of a patient's genetic predisposition to predict a favorable outcome from treatment with an immune checkpoint inhibitor.

### SUMMARY OF THE INVENTION

Provided herein are, inter alia, methods of identifying an individual having a cancer who has an increased likelihood of experiencing treatment-induced liver dysfunction during treatment comprising an immune checkpoint inhibitor (e.g., a PD-1 axis binding antagonist (e.g., an anti-PD-L1 antibody such as atezolizumab)), methods of treatment, and related articles of manufacture and compositions for use.

In one aspect, the invention provides a method of identifying a human individual having a cancer who has an increased likelihood of experiencing treatment-induced liver dysfunction during treatment comprising atezolizumab, the method comprising determining a polygenic risk score (PRS) for liver damage from a sample from the individual, wherein a PRS for liver damage that is above a liver damage reference PRS identifies the individual as one who may have an increased likelihood of experiencing treatment-induced liver dysfunction during treatment comprising atezolizumab.

In one aspect, the invention provides a method of treating a human individual having a cancer, the method comprising: (a) determining a PRS for liver damage from a sample from the individual, wherein the PRS for liver damage is above a liver damage reference PRS; (b) administering an effective amount of atezolizumab to the individual; and (c) monitoring the individual for symptoms of treatment-induced liver dysfunction.

In one aspect, the invention provides atezolizumab for use in treatment of a human individual having a cancer, the treatment comprising: (a) determining a PRS for liver damage from a sample from the individual, wherein the PRS for liver damage is above a liver damage reference PRS; (b) administering an effective amount of atezolizumab to the individual; and (c) monitoring the individual for symptoms of treatment-induced liver dysfunction.

In some aspects, the liver damage reference PRS is a pre-assigned PRS. In some aspects, the liver damage reference PRS is the first, second, third, fourth, or fifth quantile of PRSs for liver damage in a reference population. In some aspects, the reference population is a population of individuals having the cancer. In some aspects, (a) the PRS for liver damage of the sample from the individual or (b) the PRS for liver damage of a sample from an individual in the reference population is calculated using the equation:

$$\hat{S} = {\sum }_{i = 1}^{M} {β}_{i} \cdot {G}_{i}$$

wherein:
(i) Ŝ is the PRS for liver damage;
(ii) M is the number of risk alleles selected from independent genetic signals in a genome-wide association study (GWAS) for liver damage;
(iii) i represents the index of a given SNP;
(iv) βᵢ is the log odds ratio or conditionally independent odds ratio of the ith SNP; and
(v) Gᵢ={0,1,2} is the number of copies of the SNP in the sample from the individual.

In some aspects, the risk alleles are identified in the sample by whole-genome sequencing.

In some aspects, the risk alleles are the risk alleles associated with a polygenic score (PGS) having a PGS catalog identification (ID) of PGS000668, PGS002158, PGS001940, PGS000816, PGS002159, PGS000673, or PGS001941. In some aspects, the PGS catalog is available at pgscatalog.org.

In some aspects, the PRS for liver damage is a PRS for serum alanine aminotransferase (ALT) or aspartate aminotransferase (AST).

In some aspects, the sample is a whole blood sample, a plasma sample, a serum sample, or a combination thereof. In some aspects, the sample is an archival sample, a fresh sample, or a frozen sample.

In some aspects, any one of the methods described herein further comprises administering to the individual one or more additional therapeutic agents. In some aspects, the one or more additional therapeutic agents comprise paclitaxel, nab-paclitaxel, carboplatin, cisplatin, bevacizumab, pemetrexed, gemcitabine, cobimetinib, etoposide, or a combination thereof.

In some aspects, the individual has not been previously treated for the cancer. In some aspects, the individual has not been previously administered atezolizumab. In some aspects, the individual is of European ancestry.

In some aspects, the cancer is an ovarian cancer, a fallopian tube cancer, a primary peritoneal cancer, a lung cancer, a bladder cancer, a kidney cancer, breast cancer, or a skin cancer.

In some aspects, (a) the ovarian cancer is a Stage III or IV ovarian cancer; (b) the fallopian tube cancer is a Stage III or IV fallopian tube cancer; (c) the primary peritoneal cancer is a Stage III or IV primary peritoneal cancer; (d) the lung cancer is a small cell lung cancer (SCLC) or a non-small cell lung cancer (NSCLC); (e) the bladder cancer is a urothelial carcinoma (UC) or a urothelial bladder cancer (UBC); (f) the kidney cancer is a renal cell carcinoma (RCC); (g) the breast cancer is a triple negative breast cancer (TNBC); or (h) the skin cancer is a melanoma.

In some aspects, (a) the SCLC is an extensive-stage (ES) SCLC; (b) the NSCLC is a Stage IB-Stage IIIA NSCLC, a Stage IV non-squamous NSCLC or a Stage IV squamous NSCLC; (c) the UC is a high-risk muscle-invasive UC; (d) the UBC is a locally advanced or metastatic UBC; (e) the RCC is an inoperable, locally advanced, or metastatic RCC; (f) the TNBC is an early stage TNBC, a locally advanced TNBC, or a metastatic TNBC; or (g) the melanoma is a BRAFv600 wild-type melanoma.

In some aspects, in any one of the methods described herein, atezolizumab is administered as an adjuvant or a neoadjuvant therapy.

In some aspects, the treatment-induced liver dysfunction is hepatitis. In some aspects, the treatment-induced liver dysfunction is a hepatic immune-related adverse event. In some aspects, the treatment-induced liver dysfunction is determined through clinical diagnosis or measurement of laboratory values.

### BRIEF DESCRIPTION OF THE DRAWING

FIG. 1 is a set of bar graphs showing the proportion of patients for which treatment-induced liver dysfunction (i.e., hepatitis) immune-related adverse events (irAEs) were observed in atezolizumab trials (anti-PD-L1) in patients treated with either atezolizumab (“Atezo”) or control (non-atezolizumab-treated; “Non-Atezo”) for different polygenic risk score (PRS) quantiles for four different PRSs (PGS000668, PGS002158, PGS001940, PGS000816, PGS000673, PGS002159, and PGS001941), for liver damage. In particular, “0” indicates patients (i.e., the fraction of patients) without hepatitis, while “1” indicates patients (i.e., the fraction of patients) with hepatitis. For each bar, the top section of the bar indicates the fraction of “0” while the bottom section of the bar indicates the fraction of “1.”

### DETAILED DESCRIPTION OF THE INVENTION

### I. Definitions

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) aspects that are directed to that value or parameter per se. In some embodiments, reference to “about” a value or parameter herein refers to ±10% of the recited value or parameter per se. For example, description referring to “about X” includes description of “X.”

It is understood that aspects of the disclosure described herein include “comprising,” “consisting,” and “consisting essentially of” aspects.

As used herein, the term “adverse event” or “AE” refers to any unfavorable and unintended sign (including an abnormal laboratory finding), symptom, or disease temporally associated with the use of a medical treatment or procedure that may or may not be considered related to the medical treatment or procedure. Adverse events may be classified by “grade,” as defined by the National Cancer Institute Common Terminology Criteria for Adverse Events v5.0 (NIH CTCAE). In some aspects, the AE is a low grade AE, e.g., a Grade 1 or Grade 2 AE. Grade 1 includes AEs that are asymptomatic or have mild symptoms. Grade 2 includes AEs that are moderate and limit age-appropriate instrumental activities of daily living (e.g., preparing meals, shopping for groceries or clothes) and that indicate local or noninvasive intervention. In other instances, the AE is a high grade AE, e.g., a Grade 3, Grade 4, or Grade 5 AE.

Grade 3 includes AEs that are severe or medically significant, but not immediately life-threatening, and that indicate hospitalization or prolongation of hospitalization. Grade 4 includes AEs that have life-threatening consequences and indicate urgent intervention. Grade 5 includes AEs that result in or relate to death.

As used herein, the term “immune-related adverse event” or “irAE” refers to an adverse event or “adverse event of special interest” (“AESI”), as classified by the NIH CTCAE, that has a putative immune-related etiology. In some aspects, the irAE is an AESI occurring as a result of immune checkpoint inhibitor (e.g., a PD-1 axis binding antagonist (e.g., an anti-PD-L1 antibody such as atezolizumab)) therapy. In some aspects, the irAE affects liver (“liver irAE”). Liver irAEs include, but are not limited to, “immune-related hepatitis.” In some aspects, the irAE is a low grade irAE, e.g., a Grade 1 AE (Grade 1 irAE) or Grade 2 AE (Grade 2 irAE).

As used herein, the term “polygenic risk score” or “PRS” refers to a numerical value that reflects the number of single-nucleotide polymorphisms (SNPs) associated with an increased likelihood of developing a given pathological state, disease, or condition (e.g., treatment-induced liver dysfunction, e.g., hepatitis) detected in a sample (e.g., a blood sample (e.g., a whole blood sample, a plasma sample, a serum sample, a buccal swab, a tissue biopsy, or a tissue sample (e.g., a tumor tissue sample) or a combination thereof)) obtained from an individual (e.g., an individual at risk of or having a cancer). The PRS can be measured, for example, on a whole genome basis, or on the basis of a subset of the genome (e.g., a predetermined set of loci, e.g., a set of loci in linkage disequilibrium). In some aspects, the predetermined set of loci does not comprise the entire genome. In some aspects, the predetermined set of loci comprise a plurality of loci at which one or more alleles are associated with an increased risk for the given pathological state, disease, or condition. In some aspects, the predetermined set of loci comprise at least about 5 or more, about 10 or more, about 20 or more, about 50 or more, about 100 or more, about 200 or more, about 500 or more, about 1000 or more, about 2000 or more, about 5000 or more, about 10,000 or more, about 15,000 or more, or about 20,000 or more loci. In some aspects, a PRS may be referred to as a “polygenic score” or “PGS.” In some aspects, a PRS or PGS may be associated with a particular PRS or PGS ID as published in the PGS catalog (Lambert et al., Nature Genetics, 53:420-425, 2021), see e.g., pgscatalog.org).

As used herein, the term “reference polygenic risk score” or “reference PRS” refers to a PRS against which another PRS is compared, e.g., to make a diagnostic, predictive, prognostic, and/or therapeutic determination. For example, the reference PRS may be a PRS in a reference sample, a reference population, and/or a pre-determined value. In some aspects, the reference PRS is a cut-off value that significantly separates a first subset and a second subset of individuals who have been treated with an immune checkpoint inhibitor (e.g., a PD-1 axis binding antagonist (e.g., an anti-PD-L1 antibody such as atezolizumab)) in the same reference population based on a significant difference between an individual's likelihood of experiencing treatment-induced liver dysfunction (e.g., hepatitis) after treatment with an immune checkpoint inhibitor (e.g., a PD-1 axis binding antagonist (e.g., an anti-PD-L1 antibody such as atezolizumab)), at or above the cut-off value or at or below the cut-off value. In some aspects, an individual has a greater likelihood of experiencing a treatment-induced liver dysfunction (e.g., hepatitis) if the individual's PRS is at or above the cut-off value.

In some aspects, a reference PRS is defined as, e.g., the 15^{th} percentile, the 16^{th} percentile, the 17^{th} percentile, the 18^{th} percentile, the 19^{th} percentile, the 20^{th} percentile, the 21^{st} percentile, the 22^{nd} percentile, the 23^{rd} percentile, the 24^{th} percentile, the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, the 76^{th} percentile, the 77^{th} percentile, the 78^{th} percentile, the 79^{th} percentile, the 80^{th} percentile, the 81^{st} percentile, the 82^{nd} percentile, the 83^{rd} percentile, the 84^{th} percentile, or the 85^{th} percentile of PRSs in the reference population. In some aspects, a reference PRS is defined as the 50^{th} percentile of PRSs in the reference population. In some aspects, a reference PRS is defined as the median of PRSs in the reference population. In some aspects, a reference PRS is defined as the 1st, 2^{nd}, 3^{rd}, or 4^{th} quartile of PRSs in the reference population. In some aspects, a reference PRS is defined as the 1^{st}, 2^{nd}, 3^{rd}, 4^{th}, or 5^{th} quintile of PRSs in the reference population.

The term “treatment-induced liver dysfunction” refers to dysfunction in the liver or hepatic function associated with administration of a treatment or drug (e.g., atezolizumab). In some aspects, treatment-induced liver dysfunction includes adverse events (e.g., immune-related adverse events), such as elevated aspartate aminotransferase (AST) and/or alanine aminotransferase (ALT). Additional examples of treatment-induced liver dysfunction are described in Remash et al., World J Gastroenterol. 2021 Aug. 28; 27(32): 5376-5391, which is incorporated herein by reference in its entirety. In a particular aspect, a treatment-induced liver dysfunction is hepatitis.

The term “copy number of a gene” or “copy number of an allele” refers to the number of DNA loci in a cell having a particular sequence. Generally, for a given gene or locus, a mammal has two copies of each gene or locus. The copy number can be increased, e.g., by gene amplification or duplication, or reduced by deletion.

For the purposes herein, “atezolizumab” is an Fc-engineered, humanized, non-glycosylated IgG1 kappa immunoglobulin that binds PD-L1. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances (proposed INN)) List 112, Vol. 28, No. 4, 2014, p. 488.

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to bis-Fabs; Fv; Fab; Fab, Fab′-SH; F(ab′)₂; diabodies; linear antibodies; single-chain antibody molecules (e.g., scFv, scFab); and multispecific antibodies formed from antibody fragments.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2.

The heavy chain constant domains that correspond to the different classes of antibodies are called α, δ, ε, γ, and μ, respectively.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter. J. Mol. Biol. 227:381, 1991; Marks et al. J. Mol. Biol. 222:581, 1991. Also available for the preparation of human monoclonal antibodies are methods described in Cole et al. Monoclonal Antibodies and Cancer Therapy, Alan R. Liss, p. 77 (1985); Boerner et al. J. Immunol., 147(1):86-95,1991. See also van Dijk and van de Winkel. Curr. Opin. Pharmacol. 5:368-74, 2001. Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al. Proc. Natl. Acad. Sci. USA. 103:3557-3562, 2006 regarding human antibodies generated via a human B-cell hybridoma technology.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al. supra. In one aspect, for the VH, the subgroup is subgroup III as in Kabat et al. supra.

A “humanized” antibody refers to a chimeric antibody comprising amino acid residues from non-human HVRs and amino acid residues from human FRs. In certain aspects, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3).

The terms “anti-PD-L1 antibody” and “an antibody that binds to PD-L1” refer to an antibody that is capable of binding PD-L1 with sufficient affinity such that the antibody is useful as a diagnostic and/or therapeutic agent in targeting PD-L1. In one aspect, the extent of binding of an anti-PD-L1 antibody to an unrelated, non-PD-L1 protein is less than about 10% of the binding of the antibody to PD-L1 as measured, for example, by a radioimmunoassay (RIA). In some aspects, an anti-PD-L1 antibody binds to an epitope of PD-L1 that is conserved among PD-L1 from different species. In another aspect, an anti-PD-L1 antibody is atezolizumab.

As used herein, the term “immune checkpoint inhibitor” refers to a therapeutic agent that targets at least one immune checkpoint protein to alter the regulation of an immune response, e.g., down-modulating, inhibiting, up-modulating, or activating an immune response. The term “immune checkpoint blockade” may be used to refer to a therapy comprising an immune checkpoint inhibitor. Immune checkpoint proteins are known in the art and include, without limitation, programmed cell death ligand 1 (PD-L1), TIGIT, cytotoxic T-lymphocyte antigen 4 (CTLA-4), programmed cell death 1 (PD-1), programmed cell death ligand 2 (PD-L2), V-domain Ig suppressor of T cell activation (VISTA), B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, LAG-3, BTLA, IDO, OX40, and A2aR. In some aspects, an immune checkpoint protein may be expressed on the surface of an activated T cell. Therapeutic agents that can act as immune checkpoint inhibitors useful in the methods of the present invention, include, but are not limited to, therapeutic agents that target one or more of PD-L1, TIGIT, PD-1, CTLA-4, PD-L2, VISTA, B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, LAG-3, BTLA, IDO, OX40, and A2aR. In some aspects, an immune checkpoint inhibitor enhances or suppresses the function of one or more targeted immune checkpoint proteins. In some aspects, the immune checkpoint inhibitor is a PD-L1 axis binding antagonist, such as atezolizumab, as described herein.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1LG1,” “CD274,” “B7-H,” and “PDL1.”

A “blocking” antibody or an “antagonist” antibody is one which inhibits or reduces biological activity of the antigen it binds. In some aspects, blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In some aspects, the extent of binding of an antibody to an unrelated target is less than about 10% of the binding of the antibody to the target as measured, e.g., by a radioimmunoassay (RIA). In some aspects, an antibody that specifically binds to a target has a dissociation constant (K_{D}) of ≤1 μM, ≤100 nM, ≤10 nM, ≤1 nM, or ≤0.1 nM. In some aspects, an antibody specifically binds to an epitope on a protein that is conserved among the protein from different species. In other aspects, specific binding can include but does not require exclusive binding.

The term “biomarker” as used herein refers to an indicator, e.g., predictive, diagnostic, and/or prognostic, which can be detected in a sample, e.g., a single-nucleotide polymorphism (SNP), or derived therefrom (e.g., a PRS). In some aspects, a biomarker is a genetic locus, a collection of genetic loci, or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers. The biomarker may serve as an indicator of the likelihood of developing a given pathological state, disease, or condition (e.g., treatment-induced liver dysfunction (e.g., hepatitis)), or of developing a particular subtype of a disease or disorder (e.g., cancer) characterized by certain, molecular, pathological, histological.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, buccal swab, sputum, tears, perspiration, mucus, tumor lysates, and tissue culture medium, tissue extracts such as homogenized tissue, tumor tissue, cellular extracts, and combinations thereof. The sample may be an archival sample, a fresh sample, or a frozen sample. In some instances, the sample is a buccal swab, whole blood sample, a plasma sample, a serum sample, or a combination thereof.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes.

The term “survival” refers to the patient remaining alive, and includes overall survival as well as progression-free survival.

As used herein, “hazard ratio” or “HR” is a statistical definition for rates of events. For the purpose of the disclosure, hazard ratio is defined as representing the probability of an event in the experimental (e.g., treatment) group/arm divided by the probability of an event in the control group/arm at any specific point in time. An HR with a value of 1 indicates that the relative risk of an endpoint (e.g., treatment-induced liver dysfunction (e.g., hepatitis)) is equal in both the “treatment” and “control” groups; a value greater than 1 indicates that the risk is greater in the treatment group relative to the control group; and a value less than 1 indicates that the risk is greater in the control group relative to the treatment group.

The word “label” when used herein refers to a compound or composition that is conjugated or fused directly or indirectly to a reagent such as a polynucleotide probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

An “effective amount” of a compound, for example, an immune checkpoint inhibitor (e.g., a PD-1 axis binding antagonist (e.g., an anti-PD-L1 antibody such as atezolizumab)) or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The terms “cancer” and “cancerous” refer to or describe the physiological condition in mammals that is typically characterized by unregulated cell growth. Aspects of cancer include solid tumor cancers and non-solid tumor cancers. Examples of cancer include but are not limited to, carcinoma, lymphoma, blastoma, sarcoma, and leukemia or lymphoid malignancies. More particular examples of such cancers include, but are not limited to, bladder cancer (e.g., urothelial bladder cancer (UBC) or urothelial carcinoma (UC), including metastatic UC (mUC); muscle-invasive bladder cancer (MIBC), and non-muscle-invasive bladder cancer (NMIBC)); kidney or renal cancer (e.g., renal cell carcinoma (RCC)); lung cancer, including small-cell lung cancer, non-small cell lung cancer, adenocarcinoma of the lung, and squamous carcinoma of the lung; cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum (peritoneal cancer; e.g., primary peritoneal cancer; e.g., Stage III or IV primary peritoneal cancer); hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer; ovarian cancer (e.g., ovarian cancer; e.g., Stage III or IV ovarian cancer); liver cancer (e.g., hepatocellular carcinoma (HCC)); hepatoma; colon cancer; rectal cancer; colorectal cancer; endometrial or uterine carcinoma; salivary gland carcinoma; prostate cancer; fallopian tube cancer (e.g., fallopian tube cancer; e.g., Stage III or IV fallopian tube cancer); vulval cancer; thyroid cancer; hepatic carcinoma; anal carcinoma; penile carcinoma; melanoma, including superficial spreading melanoma, lentigo malignant melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myelogenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The term “bladder cancer” includes, but is not limited to, urothelial bladder cancer (UBC) and urothelial carcinoma (UC), and which may be, for example, locally advanced or metastatic. The methods described herein are suitable for treatment of various stages of cancer, including cancers that are locally advanced and/or metastatic. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage IV). In some instances, the UC is a high-risk muscle-invasive UC.

The term “breast cancer” includes, but is not limited to, HER2+ breast cancer and triple-negative breast cancer (TNBC), which is a form of breast cancer in which the cancer cells are negative for estrogen receptors (ER−), progesterone receptors (PR−), and HER2 (HER2−), and which may be locally advanced, unresectable, and/or metastatic (e.g., metastatic triple-negative breast cancer (mTNBC)).

As used herein, the terms “early TNBC” and “eTNBC” refer to early-stage TNBC, including Stage I-Stage III TNBC. Early TNBC accounts for 10% to 20% of all new early breast cancer diagnoses, with a 3-year event-free survival rate of 74% to 76% after treatment with neoadjuvant anthracycline and taxane therapy.

In some embodiments, the cancer is kidney cancer. In particular embodiments, the kidney cancer is renal cell carcinoma (RCC) (e.g., advanced RCC or metastatic RCC (mRCC), including previously untreated RCC). In some embodiments, the kidney cancer is sarcomatoid kidney cancer (e.g., sarcomatoid RCC (e.g., sarcomatoid advanced or mRCC)). In some embodiments, the RCC is an inoperable RCC.

As used herein, the term “inoperable or “unresectable” refers to a cancer for which surgical resection is not possible or cannot be safely performed.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cell proliferative disorder” and “proliferative disorder” refer to disorders that are associated with some degree of abnormal cell proliferation. In one embodiment, the cell proliferative disorder is cancer. In another embodiment, the cell proliferative disorder is a tumor.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, an immune checkpoint inhibitor (e.g., a PD-1 axis binding antagonist (e.g., an anti-PD-L1 antibody such as atezolizumab)) is used to delay development of a disease or to slow the progression of a disease.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free) (nab-paclitaxel), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the disclosure include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 A antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from GlaxoSmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), interleukin 1 (IL-1) blockers such as anakinra (Kineret), T cell costimulation blockers such as abatacept (Orencia), interleukin 6 (IL-6) blockers such as tocilizumab (ACTEMERA®); interleukin 13 (IL-13) blockers such as lebrikizumab; interferon alpha (IFN) blockers such as rontalizumab; beta 7 integrin blockers such as rhuMAb Beta7; IgE pathway blockers such as Anti-M1 prime; Secreted homotrimeric LTa3 and membrane bound heterotrimer LTa1/P2 blockers such as anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

A “growth inhibitory agent” when used herein refers to a compound or composition which inhibits growth of a cell either in vitro or in vivo. In one aspect, growth inhibitory agent is growth inhibitory antibody that prevents or reduces proliferation of a cell expressing an antigen to which the antibody binds. In another aspect, the growth inhibitory agent may be one which significantly reduces the percentage of cells in S phase. Aspects of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject or individual is a human.

As used herein, “administering” is meant a method of giving a dosage of a compound (e.g., an immune checkpoint inhibitor (e.g., a PD-1 axis binding antagonist (e.g., an anti-PD-L1 antibody such as atezolizumab))) to a subject. In some aspects, the compositions utilized in the methods herein are administered intravenously. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

The method of administration can vary depending on various factors (e.g., the compound or composition being administered and the severity of the condition, disease, or disorder being treated).

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the symptoms of the disorder being treated, the presence or size of metastases, or the size of the primary tumor.

### II. Predictive Methods and Assays

The invention is based, at least in part, on the discovery that a PRS for liver damage can be used as a biomarker (e.g., a predictive biomarker) in the treatment of an individual having a cancer, e.g., for determining whether an individual having such a cancer is likely to experience treatment-induced liver dysfunction during treatment with an immune checkpoint inhibitor (e.g., a PD-1 axis binding antagonist (e.g., an anti-PD-L1 antibody such as atezolizumab)) or for selecting a therapy for an individual having a cancer. In some aspects, a high PRS for liver damage is associated with increased likelihood of experiencing treatment-induced liver dysfunction (e.g., hepatitis) during treatment with an immune checkpoint inhibitor (e.g., the PD-1 axis binding antagonist (e.g., the anti-PD-L1 antibody such as atezolizumab)). See, e.g., Examples 1 and 2.

Accordingly, also provided herein are methods and assays of evaluating PRSs for liver damage in a sample from an individual. Any of the methods provided herein may include administering an immune checkpoint inhibitor (e.g., a PD-1 axis binding antagonist (e.g., an anti-PD-L1 antibody such as atezolizumab)) to the individual. Any of the methods may further include administering an effective amount of an additional therapeutic agent, as described herein, to the individual.

### A. Diagnostic Methods and Assays

i. Methods of Determining Polygenic Risk Scores (PRSs)
Ia. Identification of Risk Alleles

In some aspects, the invention features methods that include determining one or more polygenic risk scores (PRSs) of an individual, e.g., PRSs for liver damage. PRS may be represented as the number of single-nucleotide polymorphisms (SNPs) associated with increased likelihood of having or developing a disease, state, or condition (“risk alleles”), e.g., liver damage risk alleles counted over a defined number of sequenced base pairs or in the whole genome sequence of an individual.

Risk alleles may be identified using a number of methods. In some aspects, risk alleles may be identified in a genome-wide association study (GWAS) for a pathological state, disease, or condition of interest. In some aspects, individuals included in the GWAS may be clinically diagnosed as having the disease, state, or condition, e.g., diagnosed using the International Classification of Diseases (ICD) code. In other aspects, individuals included in the GWAS may self-identify as having the disease, state, or condition. GWAS may identify one or more genic or non-genic loci (e.g., a SNP), e.g., 1 or more loci, 5 or more loci, 10 or more loci, 15 or more loci, 20 or more loci, 25 or more loci, 30 or more loci, 40 or more loci, 50 or more loci, 60 or more loci, 70 or more loci, 80 or more loci, 90 or more loci, 100 or more loci, 150 or more loci, 200 or more loci, 300 or more loci, 400 or more loci, 500 or more loci, 1000 or more loci, 2000 or more loci, 3000 or more loci, 4000 or more loci, 5000 or more loci, 10,000 or more loci, 50,000 or more loci, 100,000 or more loci, 200,000 or more loci, or 500,000 or more loci to be included in the set of risk alleles. The GWAS p-value threshold at which the PRS is most predictive is often unknown, and PRSs may use SNPs that do not achieve genome-wide significant p-values in the original GWAS (Dudbridge, PLoS Genet., 9: e1003348, 2013; Euesden et al., Bioinformatics, 31: 1466-1468, 2015). The p-value threshold for inclusion in the set of risk alleles may be, e.g., p<0.2, p<0.1, p<0.05, p<0.01, p<0.001, p<1×10⁻⁴, p<1×10⁻⁵, p<1×10⁻⁶, p<1×10⁻⁷, p<1×10⁻⁸, p<1×10⁻⁹, or p<1×10⁻¹⁰.

In some aspects, the GWAS may identify risk alleles for liver damage.

In some aspects, the GWAS for liver damage identifies 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 6,000 or more, 10,000 or more, 15,000 or more, 25,000 or more, 50,000 or more, 100,000 or more, 150,000 or more, or 200,000 or more risk alleles for liver damage. In some aspects, the GWAS for liver damage identifies 70 to 110,000 risk alleles for liver damage, e.g., 100 to 100,000 risk alleles, 250 to 150,000 risk alleles, 500 to 100,000 risk alleles, 1000 to 50,000 risk alleles, 2000 to 25,000 risk alleles, 3000 to 20,000 risk alleles. 4,000 to 15,000 risk alleles, or 5,000 to 10,000 risk alleles.

Ib. Determination of Individual PRS

In some aspects, the PRS of an individual is represented as the number of SNPs associated with risk for liver damage (“risk alleles”) occurring in the individual as counted over a defined number of sequenced base pairs. In some aspects, the number of sequenced base pairs (bp) is, e.g., at least 50 bp, at least 100 bp, at least 500 bp, at least 1 kbp, at least 10 kbp, at least 50 kbp, at least 100 kbp, at least 500 kbp, at least 1000 kbp, at least 1 Mbp, at least 500 Mbp, or at least 1 Gbp. In other aspects, the sequenced base pairs comprise the whole genome sequence (WGS) or whole exome sequence (WES) of an individual. In particular aspects, the sequenced base pairs comprise the WGS of an individual. Methods for WGS include, but are not limited to, the Illumina X10 HISEQ® platform. In some aspects, WGS data is generated to an average read depth of at least 2×, at least 5×, at least 10×, at least 15×, at least 20×, at least 25×, at least 30×, at least 35×, at least 40×, at least 45×, at least 50×, or at least 100× coverage. Reads may be mapped to a reference genome, e.g., a human reference genome, e.g., hg38/GRCh38 (GCA_000001405.15). See, for example, Van der Auwera et al., Curr Protoc Bioinformatics, 11: 11.10.1-11.10.33, 2013; McKenna et al., Genome Res., 20: 1297-1303, 2010; and DePristo et al., Nat. Genet., 43: 491-498, 2011.

In some aspects, the risk alleles are risk alleles that affect serum alanine aminotransferase (ALT), aspartate aminotransferase (AST), gamma-glutamyl Transferase (GGT), and/or alkaline phosphatase (ALP) levels or are associated with risk of cirrhosis of liver. In a preferred aspect, the risk alleles are risk alleles that affect serum alanine aminotransferase (ALT) measurement or aspartate aminotransferase (AST) levels. In some aspects, the PRSs may include one or more of 16 PRSs for ALT (PGS000668, PGS002158, PGS001940, PGS000816), AST (PGS000673, PGS002159, PGS001941), GGT (PGS001964, PGS002182, PGS000683, PGS000817), ALP (PGS000815, PGS000670, PGS001939, PGS002157), and cirrhosis of liver (PGS000726) that are available from the PGS catalog (Lambert et al., Nature Genetics, 53:420-425, 2021).

In some aspects, the risk alleles are the risk alleles associated with a polygenic score (PGS) having a PGS catalog ID of PGS000668, PGS002158, PGS001940, PGS000816, PGS000673, PGS002159, PGS001941, PGS001964, PGS002182, PGS000683, PGS000817, PGS000815, PGS000670, PGS001939, PGS002157, or PGS000726. In in a preferred aspect, the risk alleles are the risk alleles associated with a PGS having a PGS catalog ID of PGS000668, PGS002158, PGS001940, PGS000816, PGS002159, PGS000673, or PGS001941. In some aspects, the PGS ID is defined in the PGS catalog (pgscatalog.org).

PRSs may be assessed in one or more samples from an individual. Any suitable sample may be used. For example, the sample may be a tissue sample, a primary or cultured cell or cell line, a cell supernatants a cell lysate, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, buccal swab, sputum, tears, perspiration, mucus, tumor lysates, tissue culture medium, a tissue extract such as homogenized tissue, tumor tissue, a cellular extract, and any combination thereof. In some aspects, a sample contains germline DNA. In some aspects, the sample is a formalin-fixed and paraffin-embedded (FFPE) sample, an archival sample, a fresh sample, or a frozen sample.

In some aspects, a PRS for liver damage may be determined for a sample from an individual. In some aspects, the PRS identifies 0, 1 or more, 5 or more, 10 or more, 15 or more, 20 or more, 25 or more, 30 or more, 40 or more, 50 or more, 60 or more, 70 or more, 80 or more, 90 or more, 100 or more, 150 or more, 200 or more, 300 or more, 400 or more, 500 or more, 1000 or more, 2000 or more, 3000 or more, 4000 or more, 5000 or more, 10,000 or more, 50,000 or more, 100,000 or more, 200,000 or more, or 500,000 or more risk alleles for liver damage in the sample from the individual. In some aspects, the PRS for liver damage of the individual is higher than 0%, higher than 10%, higher than 20%, higher than 30%, higher than 40%, higher than 50%, higher than 60%, higher than 70%, higher than 80%, higher than 90%, or higher than 100% of PRSs for liver damage for individuals in a reference population. In some aspects, the PRS for liver damage of the individual may be higher than a 1^{st} quintile, a 2^{nd} quintile, a 3^{rd} quintile, a 4^{th} quintile, or a 5^{th} quintile of PRSs for liver damage for individuals in a reference population. In some aspects, the PRS for liver damage of the individual may be higher than a 1^{st} quartile, a 2^{nd} quartile, a 3^{rd} quartile, or a 4^{th} quartile of PRSs for liver damage for individuals in a reference population. In some aspects, the PRS for liver damage of the individual may be higher than a median of PRSs for liver damage for individuals in a reference population.

In one aspect, the PRS of an individual for liver damage is represented as the number of SNPs associated with risk for liver damage or treatment-induced liver dysfunction (e.g., hepatitis) counted in a WGS sample, wherein the sample is a blood sample, e.g., a whole blood sample, a plasma sample, a serum sample, or a combination thereof.

In some aspects, the PRS for liver damage of the sample from the individual or the PRS for liver damage of a sample from an individual in the reference population is calculated using the equation:

$$\hat{S} = {\sum }_{i = 1}^{M} {β}_{i} \cdot {G}_{i}$$

wherein Ŝ is the PRS for liver damage; M is the number of risk alleles selected from independent genetic signals in a genome-wide association study (GWAS) for liver damage; i represents the index of a given SNP; βᵢ is the log odds ratio or conditionally independent odds ratio of the ith SNP; and Gᵢ={0,1,2} is the number of copies of the SNP in the sample from the individual.

In some embodiments, M is the number of independent signals in the GWAS after fine mapping, Ri corresponds to the conditional effect size for the variant with the highest PPA for ith signal, and Gᵢ={0,1,2} corresponds to the number of copies of the risk allele.

ic. Reference Populations

In some aspects, a PRS of an individual for liver damage is compared to a PRS in a reference population. In some aspects, the reference population is a population of individuals having a cancer (e.g., an ovarian cancer, a fallopian tube cancer, a primary peritoneal cancer, a lung cancer, a bladder cancer, a kidney cancer, breast cancer, or a skin cancer), the population of individuals consisting of a first subset of individuals who have been treated with an immune checkpoint inhibitor (e.g., a PD-1 axis binding antagonist (e.g., an anti-PD-L1 antibody such as atezolizumab)) and/or a second subset of individuals who have been treated with a non-immune-checkpoint-inhibitor therapy, e.g., a chemotherapy, wherein the non-immune-checkpoint-inhibitor therapy does not comprise an immune checkpoint inhibitor (e.g., the PD-1 axis binding antagonist (e.g., the anti-PD-L1 antibody such as atezolizumab)). In other aspects, the reference population is the GWAS population. In some aspects, the reference population may be used to determine a liver damage reference PRS and/or a liver damage reference PRS.

In some aspects, the liver damage reference PRS is defined as, e.g., the 0^{th} percentile (i.e., the 1^{st} quartile or the 1^{st} quintile), 1st percentile, 2^{nd} percentile, 3^{rd} percentile, 4^{th} percentile, 5^{th} percentile, 6^{th} percentile, 7^{th} percentile, 8^{th} percentile, 9^{th} percentile, 10^{th} percentile, 11^{th} percentile, 12^{th} percentile, 13^{th} percentile, 14^{th} percentile, 15^{th} percentile, 16^{th} percentile, 17^{th} percentile, 18^{th} percentile, 19^{th} percentile, 20^{th} percentile (i.e., the 2^{nd} quintile), 21^{st} percentile, 22^{nd} percentile, 23^{rd} percentile, 24^{th} percentile, 25^{th} percentile (i.e., the 2^{nd} quartile), 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile (i.e., the 3^{rd} quintile), 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile (i.e., the median or the 3^{rd} quartile), 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile (i.e., the 4^{th} quintile), 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile (i.e., the 4^{th} quartile), 76^{th} percentile, 77^{th} percentile, 78^{th} percentile, 79^{th} percentile, 80^{th} percentile (i.e., the 5^{th} quintile), 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of polygenic risk scores (PRSs) for liver damage in the reference population (e.g., the PRSs of the individuals of the reference population).

In some aspects, the liver damage reference PRS is defined as the 25^{th} percentile of PRSs for liver damage in the reference population. In some aspects, the liver damage reference PRS is defined as the 50^{th} percentile of PRSs for liver damage in the reference population. In some aspects, the liver damage reference PRS is defined as the median of PRSs for liver damage in the reference population. In some aspects, the liver damage reference PRS is defined as the 75^{th} percentile of PRSs for liver damage in the reference population. In some aspects, the liver damage reference PRS is defined as the 1st quintile, 2^{nd} quintile, 3^{rd} quintile, 4^{th} quintile, or 5^{th} quintile of PRSs for liver damage for individuals in a reference population. In some aspects, the liver damage reference PRS is defined as the 1st quartile, 2^{nd} quartile, 3^{rd} quartile, or 4^{th} quartile of PRSs for liver damage for individuals in a reference population. In some aspects, the PRS for liver damage of the individual may be higher than a median of PRSs for liver damage for individuals in a reference population. In some aspects, the PRS for liver damage of the individual may be higher than a 1st quintile, a 2^{nd} quintile, a 3^{rd} quintile, a 4^{th} quintile, or a 5^{th} quintile of PRSs for liver damage for individuals in a reference population. In some aspects, the PRS for liver damage of the individual may be higher than a 1st quartile, a 2^{nd} quartile, a 3^{rd} quartile, or a 4^{th} quartile of PRSs for liver damage for individuals in a reference population.

B. Methods of Identifying Patients Likely to Experience Liver-Related irAEs (e.g., Hepatitis)

In some aspects, the invention features a method of identifying an individual having a cancer (e.g., an ovarian cancer, a fallopian tube cancer, a primary peritoneal cancer, a lung cancer, a bladder cancer, a kidney cancer, breast cancer, or a skin cancer) who has an increased likelihood of experiencing treatment-induced liver dysfunction during treatment comprising an immune checkpoint inhibitor (e.g., a PD-1 axis binding antagonist (e.g., an anti-PD-L1 antibody such as atezolizumab)), the method comprising determining a polygenic risk score (PRS) for liver damage from a sample from the individual, wherein a PRS for liver damage that is above a liver damage reference PRS identifies the individual as one who may have an increased likelihood of experiencing treatment-induced liver dysfunction during treatment comprising an immune checkpoint inhibitor (e.g., the PD-1 axis binding antagonist (e.g., the anti-PD-L1 antibody such as atezolizumab)). In some aspects, the treatment-induced liver dysfunction is hepatitis. In some aspects, the treatment-induced liver dysfunction is determined through clinical diagnosis or measurement of laboratory values.

In some aspects, the invention features a method of selecting an individual having a cancer (e.g., an ovarian cancer, a fallopian tube cancer, a primary peritoneal cancer, a lung cancer, a bladder cancer, a kidney cancer, breast cancer, or a skin cancer) for treatment comprising an immune checkpoint inhibitor (e.g., a PD-1 axis binding antagonist (e.g., an anti-PD-L1 antibody such as atezolizumab)), the method comprising determining a polygenic risk score (PRS) for liver damage from a sample from the individual, wherein a PRS for liver damage that is above a liver damage reference PRS identifies the individual as one who may have an increased likelihood of experiencing treatment-induced liver dysfunction during treatment comprising an immune checkpoint inhibitor (e.g., the PD-1 axis binding antagonist (e.g., the anti-PD-L1 antibody such as atezolizumab)). In some aspects, the treatment-induced liver dysfunction is hepatitis. In some aspects, the treatment-induced liver dysfunction is determined through clinical diagnosis or measurement of laboratory values.

In some aspects, the invention features a method of selecting a therapy for an individual having a cancer (e.g., an ovarian cancer, a fallopian tube cancer, a primary peritoneal cancer, a lung cancer, a bladder cancer, a kidney cancer, breast cancer, or a skin cancer) for treatment comprising an immune checkpoint inhibitor (e.g., a PD-1 axis binding antagonist (e.g., an anti-PD-L1 antibody such as atezolizumab)), the method comprising determining a polygenic risk score (PRS) for liver damage from a sample from the individual, wherein a PRS for liver damage that is above a liver damage reference PRS identifies the individual as one who may have an increased likelihood of experiencing treatment-induced liver dysfunction during treatment comprising an immune checkpoint inhibitor (e.g., the PD-1 axis binding antagonist (e.g., the anti-PD-L1 antibody such as atezolizumab)). In some aspects, the treatment-induced liver dysfunction is hepatitis. In some aspects, the treatment-induced liver dysfunction is determined through clinical diagnosis or measurement of laboratory values.

In some aspects, the PRS of the individual for liver damage is greater than 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of PRSs for liver damage in the reference population.

In some aspects, the liver damage reference PRS is defined as the median of PRSs for liver damage in the reference population, and the PRS for liver damage of the individual is greater than the median of PRSs for liver damage in the reference population. In some aspects, the liver damage reference PRS is defined as the 1st quintile, 2^{nd} quintile, 3^{rd} quintile, 4^{th} quintile, or 5^{th} quintile of PRSs for liver damage in the reference population, and the PRS for liver damage of the individual is greater than the 1st quintile, 2^{nd} quintile, 3^{rd} quintile, 4^{th} quintile, or 5^{th} quintile of PRSs for liver damage in the reference population. In some aspects, the liver damage reference PRS is defined as the 1^{st} quartile, 2^{nd} quartile, 3^{rd} quartile, or 4^{th} quartile of PRSs for liver damage in the reference population, and the PRS for liver damage of the individual is greater than the 1st quartile, 2^{nd} quartile, 3^{rd} quartile, or 4^{th} quartile of PRSs for liver damage in the reference population.

### III. Methods of Treatment

In some aspects, the invention features a method of treating an individual having a cancer (e.g., an ovarian cancer, a fallopian tube cancer, a primary peritoneal cancer, a lung cancer, a bladder cancer, a kidney cancer, breast cancer, or a skin cancer), the method comprising (a) determining a PRS for liver damage from a sample from the individual, wherein the PRS for liver damage is above a liver damage reference PRS; (b) administering an effective amount of an immune checkpoint inhibitor (e.g., a PD-1 axis binding antagonist (e.g., an anti-PD-L1 antibody such as atezolizumab)) to the individual; and (c) monitoring the individual for symptoms of treatment-induced liver dysfunction. In some aspects, the treatment-induced liver dysfunction is hepatitis. In some aspects, the treatment-induced liver dysfunction is determined through clinical diagnosis or measurement of laboratory values.

In other aspects, the invention features a method of treating an individual having a cancer (e.g., an ovarian cancer, a fallopian tube cancer, a primary peritoneal cancer, a lung cancer, a bladder cancer, a kidney cancer, breast cancer, or a skin cancer), the method comprising administering an effective amount of an immune checkpoint inhibitor (e.g., a PD-1 axis binding antagonist (e.g., an anti-PD-L1 antibody such as atezolizumab)) to the individual, wherein a PRS for liver damage from a sample from the individual has been determined to be above a liver damage reference PRS. In some aspects, the treatment-induced liver dysfunction is hepatitis. In some aspects, the treatment-induced liver dysfunction is determined through clinical diagnosis or measurement of laboratory values.

In yet other aspects, the invention features a method of treating an individual having a cancer (e.g., an ovarian cancer, a fallopian tube cancer, a primary peritoneal cancer, a lung cancer, a bladder cancer, a kidney cancer, breast cancer, or a skin cancer), the method comprising administering an effective amount of an immune checkpoint inhibitor (e.g., a PD-1 axis binding antagonist (e.g., an anti-PD-L1 antibody such as atezolizumab)) to the individual, wherein the patient has been monitored for symptoms of liver dysfunction based on a PRS for liver damage from a sample from the individual being above a liver damage reference PRS. In some aspects, the treatment-induced liver dysfunction is hepatitis. In some aspects, the treatment-induced liver dysfunction is determined through clinical diagnosis or measurement of laboratory values.

In some aspects, the individual is monitored for symptoms of treatment-induced liver dysfunction prior to and periodically during treatment with an immune checkpoint inhibitor (e.g., a PD-1 axis binding antagonist (e.g., an anti-PD-L1 antibody such as atezolizumab)). In some aspects, treatment with an immune checkpoint inhibitor (e.g., the PD-1 axis binding antagonist (e.g., the anti-PD-L1 antibody such as atezolizumab)) is continued in the case of hepatitis based on the severity (e.g., interrupted in the case of severe hepatitis).

In some aspects, the treatment-induced liver dysfunction is determined through clinical diagnosis or measurement of laboratory values.

### A. Cancers

In some aspects, an immune checkpoint inhibitor (e.g., a PD-1 axis binding antagonist (e.g., an anti-PD-L1 antibody such as atezolizumab)) is used to treat or delay progression of a cancer in a subject in need thereof. In some aspects, the subject is a human. Examples of cancer include but are not limited to, carcinoma, lymphoma, blastoma, sarcoma, and leukemia or lymphoid malignancies. More particular examples of such cancers include, but are not limited to, bladder cancer (e.g., urothelial bladder cancer (UBC) or urothelial carcinoma (UC), including metastatic UC (mUC); muscle-invasive bladder cancer (MIBC), and non-muscle-invasive bladder cancer (NMIBC)); kidney or renal cancer (e.g., renal cell carcinoma (RCC)); lung cancer, including small-cell lung cancer (SCLC) (e.g., extensive-stage SCLC), non-small cell lung cancer (NSCLC) (e.g., non-squamous NSCLC or squamous NSCLC), adenocarcinoma of the lung, and squamous carcinoma of the lung; cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum (peritoneal cancer; e.g., primary peritoneal cancer; e.g., Stage III or IV primary peritoneal cancer); hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer; ovarian cancer (e.g., ovarian cancer; e.g., Stage III or IV ovarian cancer); liver cancer (e.g., hepatocellular carcinoma (HCC)); hepatoma; colon cancer; rectal cancer; colorectal cancer; endometrial or uterine carcinoma; salivary gland carcinoma; prostate cancer; fallopian tube cancer (e.g., fallopian tube cancer; e.g., Stage III or IV fallopian tube cancer); vulval cancer; thyroid cancer; hepatic carcinoma; anal carcinoma; penile carcinoma; melanoma, including superficial spreading melanoma, lentigo malignant melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myelogenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

In some aspects, bladder cancer includes urothelial bladder cancer (UBC) and urothelial carcinoma (UC), and which may be, for example, locally advanced or metastatic. The methods described herein are suitable for treatment of various stages of cancer, including cancers that are locally advanced and/or metastatic. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage IV). In some instances, the UC is a high-risk muscle-invasive UC).

In some aspects, breast cancer includes, but is not limited to, HER2+ breast cancer and triple-negative breast cancer (TNBC), which is a form of breast cancer in which the cancer cells are negative for estrogen receptors (ER−), progesterone receptors (PR−), and HER2 (HER2−), and which may be locally advanced, unresectable, and/or metastatic (e.g., metastatic triple-negative breast cancer (mTNBC)). TNBC may be early TNBC (eTNBC) or early-stage TNBC, including Stage I-Stage III TNBC.

In some aspects, the cancer is kidney cancer. In particular aspects, the kidney cancer is renal cell carcinoma (RCC) (e.g., advanced RCC or metastatic RCC (mRCC), including previously untreated RCC). In some aspects, the kidney cancer is sarcomatoid kidney cancer (e.g., sarcomatoid RCC (e.g., sarcomatoid advanced or mRCC)). In some aspects, the RCC is an inoperable RCC. In some aspects, the kidney cancer is a sarcomatoid.

In some aspects, the cancer is lung cancer. In some aspects, the lung cancer is NSCLC. In some aspects, the NSCLS is Stage IV non-squamous NSCLC. In some aspects, the NSCLS is Stage IV squamous NSCLC. In some aspects, the NSCLC is chemotherapy-naive Stage IV non-squamous NSCLC. In some aspects, the NSCLC is Stage IB-Stage IIIA NSCLC. In some aspects, the lung cancer is SCLC. In some aspects, the SCLC is untreated extensive-stage SCLC.

### B. Methods of Delivery

The compositions utilized in the methods described herein (e.g., comprising an immune checkpoint inhibitor (e.g., a PD-1 axis binding antagonist (e.g., an anti-PD-L1 antibody such as atezolizumab))) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some aspects, an immune checkpoint inhibitor (e.g., a PD-1 axis binding antagonist (e.g., an anti-PD-L1 antibody such as atezolizumab)) is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

An immune checkpoint inhibitor (e.g., a PD-1 axis binding antagonist (e.g., an anti-PD-L1 antibody such as atezolizumab)) described herein (and any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. An immune checkpoint inhibitor (e.g., the PD-1 axis binding antagonist (e.g., the anti-PD-L1 antibody such as atezolizumab)) need not be, but is optionally formulated with and/or administered concurrently with one or more agents currently used to prevent or treat the disorder in question, e.g., one or more of the agents provided in Section IIIC herein. The effective amount of such other agents may depend on the amount of an immune checkpoint inhibitor (e.g., the PD-1 axis binding antagonist (e.g., the anti-PD-L1 antibody such as atezolizumab)) present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the treatment of a cancer, e.g., a cancer described in Section IIIA herein, the appropriate dosage of an immune checkpoint inhibitor (e.g., a PD-1 axis binding antagonist (e.g., an anti-PD-L1 antibody such as atezolizumab)) (when used alone or in combination with one or more other additional therapeutic agents) will depend on the type of disease to be treated, the severity and course of the disease, whether an immune checkpoint inhibitor (e.g., the PD-1 axis binding antagonist (e.g., the anti-PD-L1 antibody such as atezolizumab)) is administered for preventive or therapeutic purposes, previous therapy, the patient's clinical history and response to an immune checkpoint inhibitor (e.g., the PD-1 axis binding antagonist (e.g., the anti-PD-L1 antibody such as atezolizumab)), and the discretion of the attending physician. An immune checkpoint inhibitor (e.g., a PD-1 axis binding antagonist (e.g., an anti-PD-L1 antibody such as atezolizumab)) is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the immune checkpoint inhibitor). An initial higher loading dose, followed by one or more lower doses, may be administered. However, other dosage regimens may be useful. The progress of this therapy is easily monitored by conventional techniques and assays. In some aspects, an immune checkpoint inhibitor (e.g., a PD-1 axis binding antagonist (e.g., an anti-PD-L1 antibody such as atezolizumab)) may be administered as an adjuvant therapy. In some aspects, an immune checkpoint inhibitor (e.g., a PD-1 axis binding antagonist (e.g., an anti-PD-L1 antibody such as atezolizumab)) may be administered as a neoadjuvant therapy.

For example, as a general proposition, the therapeutically effective amount of an immune checkpoint inhibitor (e.g., a PD-1 axis binding antagonist (e.g., an anti-PD-L1 antibody such as atezolizumab)) administered to human will be in the range of about 0.01 to about 50 mg/kg of patient body weight, whether by one or more administrations. In some aspects, the antibody used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or monthly, for example. In some aspects, an immune checkpoint inhibitor (e.g., the PD-1 axis binding antagonist (e.g., the anti-PD-L1 antibody such as atezolizumab)) is administered at 15 mg/kg. However, other dosage regimens may be useful. In one aspect, atezolizumab is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, or about 1800 mg on day 1 of 21-day cycles (every three weeks, q3w). In some aspects, atezolizumab is administered at 1200 mg intravenously every three weeks (q3w). In some aspects, atezolizumab is administered at a fixed dose of about 840 mg every two weeks (q2w). In some aspects, atezolizumab is administered at a fixed dose of about 1680 mg every four weeks (q4w). The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

In some aspects, the individual has not been previously treated for the cancer. For example, in some aspects, the treatment is a first-line treatment. In other aspects, the individual has received at least one prior anti-cancer therapy. For example, in some aspects, the treatment is a second-line (2L), third-line (3L), fourth-line (4L), fifth-line (5L), or later treatment. In some aspects, the individual has not been previously administered an immune checkpoint inhibitor (e.g., the PD-1 axis binding antagonist (e.g., the anti-PD-L1 antibody such as atezolizumab)). In some aspects, the individual has not been previously administered atezolizumab.

In some aspects, the individual is a human. In some aspects, the individual is of European ancestry.

### C. Additional Therapeutic Agents

In some aspects, an immune checkpoint inhibitor (e.g., a PD-1 axis binding antagonist (e.g., an anti-PD-L1 antibody such as atezolizumab)) is used with one or more additional therapeutic agents, e.g., in a combination therapy. In some aspects, the composition comprising an immune checkpoint inhibitor (e.g., a PD-1 axis binding antagonist (e.g., an anti-PD-L1 antibody such as atezolizumab)) further comprises the additional therapeutic agent. In another aspect, the additional therapeutic agent is delivered in a separate composition. In some aspects, the one or more additional therapeutic agents comprise an immunomodulatory agent, an anti-neoplastic agent, a chemotherapeutic agent, a growth inhibitory agent, an anti-angiogenic agent, a radiation therapy, a cytotoxic agent, a cell-based therapy, or a combination thereof.

Combination therapies as described above encompass combined administration (wherein two or more therapeutic agents are included in the same or separate formulations) and separate administration (wherein administration of an immune checkpoint inhibitor (e.g., a PD-1 axis binding antagonist (e.g., an anti-PD-L1 antibody such as atezolizumab)) can occur prior to, simultaneously, and/or following, administration of the additional therapeutic agent or agents). In one aspect, administration of an immune checkpoint inhibitor (e.g., a PD-1 axis binding antagonist (e.g., an anti-PD-L1 antibody such as atezolizumab)) and administration of an additional therapeutic agent occur within about one month, or within about one, two or three weeks, or within about one, two, three, four, five, or six days, of each other.

i. Chemotherapeutic Agents

In some aspects, the additional therapeutic agent is a chemotherapeutic agent. A chemotherapeutic agent is a chemical compound useful in the treatment of cancer. Exemplary chemotherapeutic agents include, but are not limited to erlotinib (TARCEVA®, Genentech/OSI Pharm.), anti-hormonal agents that act to regulate or inhibit hormone action on tumors such as anti-estrogens and selective estrogen receptor modulators (SERMs), antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), ortrastuzumab (HERCEPTIN®, Genentech), EGFR inhibitors (EGFR antagonists), tyrosine kinase inhibitors, and chemotherapeutic agents also include non-steroidal anti-inflammatory drugs (NSAIDs) with analgesic, antipyretic and anti-inflammatory effects.

ii. Growth Inhibitory Agents

In some aspects, the additional therapeutic agent is a growth inhibitory agent. Exemplary growth inhibitory agents include agents that block cell cycle progression at a place other than S phase, e.g., agents that induce G1 arrest (e.g., DNA alkylating agents such as tamoxifen, prednisone, dacarbazine, mechlorethamine, cisplatin, methotrexate, 5-fluorouracil, or ara-C) or M-phase arrest (e.g., vincristine, vinblastine, taxanes (e.g., paclitaxel, nab-paclitaxel, and docetaxel), doxorubicin, epirubicin, daunorubicin, etoposide, or bleomycin).

iii. Radiation Therapies

In some aspects, the additional therapeutic agent is a radiation therapy. Radiation therapies include the use of directed gamma rays or beta rays to induce sufficient damage to a cell so as to limit its ability to function normally or to destroy the cell altogether. Typical treatments are given as a one-time administration and typical dosages range from 10 to 200 units (Grays) per day.

iv. Cytotoxic Agents

In some aspects, the additional therapeutic agent is a cytotoxic agent, e.g., a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and antitumor or anticancer agents.

In some instances, the methods include administering to the individual an anti-cancer therapy other than, or in addition to, an immune checkpoint inhibitor (e.g., a PD-1 axis binding antagonist (e.g., an anti-PD-L1 antibody such as atezolizumab)) (e.g., an anti-neoplastic agent, a chemotherapeutic agent, a growth inhibitory agent, an anti-angiogenic agent, a radiation therapy, or a cytotoxic agent).

In some instances, the methods further involve administering to the patient an effective amount of an additional therapeutic agent. In some instances, the additional therapeutic agent is selected from the group consisting of an anti-neoplastic agent, a chemotherapeutic agent, a growth inhibitory agent, an anti-angiogenic agent, a radiation therapy, a cytotoxic agent, and combinations thereof. In some instances, an immune checkpoint inhibitor (e.g., a PD-1 axis binding antagonist (e.g., an anti-PD-L1 antibody such as atezolizumab)) may be administered in conjunction with a chemotherapy or chemotherapeutic agent. In some instances, an immune checkpoint inhibitor (e.g., a PD-1 axis binding antagonist (e.g., an anti-PD-L1 antibody such as atezolizumab)) may be administered in conjunction with a radiation therapy agent. In some instances, an immune checkpoint inhibitor (e.g., a PD-1 axis binding antagonist (e.g., an anti-PD-L1 antibody such as atezolizumab)) may be administered in conjunction with a targeted therapy or targeted therapeutic agent. In some instances, an immune checkpoint inhibitor (e.g., a PD-1 axis binding antagonist (e.g., an anti-PD-L1 antibody such as atezolizumab)) may be administered in conjunction with an immunotherapy or immunotherapeutic agent, for example a monoclonal antibody. In some instances, the additional therapeutic agent is an agonist directed against a co-stimulatory molecule. In some instances, the additional therapeutic agent is an antagonist directed against a co-inhibitory molecule. In some instances, an immune checkpoint inhibitor (e.g., the PD-1 axis binding antagonist (e.g., the anti-PD-L1 antibody such as atezolizumab)) is administered as a monotherapy.

In some aspects, the additional therapeutic agent is carboplatin. In some aspects, the additional therapeutic agent is paclitaxel. In some aspects, the additional therapeutic agent is nab-paclitaxel. In some aspects, the additional therapeutic agent is cobimetinib. In some aspects, the additional therapeutic agent is pemetrexed. In some aspects, the additional therapeutic agent is gemcitabine. In some aspects, the additional therapeutic agent is cisplatin. In some aspects, the additional therapeutic agent is bevacizumab. In some aspects, the additional therapeutic agent is sunitinib. In some aspects, the additional therapeutic agent is etoposide. In some aspects, the additional therapeutic agent is doxorubicin. In some aspects, the additional therapeutic agent is cyclophosphamide.

Without wishing to be bound to theory, it is thought that enhancing T-cell stimulation, by promoting a co-stimulatory molecule or by inhibiting a co-inhibitory molecule, may promote tumor cell death thereby treating or delaying progression of cancer. In some instances, an immune checkpoint inhibitor (e.g., a PD-1 axis binding antagonist (e.g., an anti-PD-L1 antibody such as atezolizumab)) may be administered in conjunction with an agonist directed against a co-stimulatory molecule. In some instances, a co-stimulatory molecule may include CD40, CD226, CD28, OX40, GITR, CD137, CD27, HVEM, or CD127. In some instances, the agonist directed against a co-stimulatory molecule is an agonist antibody that binds to CD40, CD226, CD28, OX40, GITR, CD137, CD27, HVEM, or CD127. In some instances, an immune checkpoint inhibitor (e.g., a PD-1 axis binding antagonist (e.g., an anti-PD-L1 antibody such as atezolizumab)) may be administered in conjunction with an antagonist directed against a co-inhibitory molecule. In some instances, a co-inhibitory molecule may include CTLA-4 (also known as CD152), TIM-3, BTLA, VISTA, LAG-3, B7-H3, B7-H4, IDO, TIGIT, MICA/B, or arginase. In some instances, the antagonist directed against a co-inhibitory molecule is an antagonist antibody that binds to CTLA-4, TIM-3, BTLA, VISTA, LAG-3, B7-H3, B7-H4, IDO, TIGIT, MICA/B, or arginase.

In some instances, an immune checkpoint inhibitor (e.g., a PD-1 axis binding antagonist (e.g., an anti-PD-L1 antibody such as atezolizumab)) may be administered in conjunction with an antagonist directed against CTLA-4 (also known as CD152), e.g., a blocking antibody. In some instances, an immune checkpoint inhibitor (e.g., a PD-1 axis binding antagonist (e.g., an anti-PD-L1 antibody such as atezolizumab)) may be administered in conjunction with ipilimumab (also known as MDX-010, MDX-101, or YERVOY®). In some instances, an immune checkpoint inhibitor (e.g., a PD-1 axis binding antagonist (e.g., an anti-PD-L1 antibody such as atezolizumab)) may be administered in conjunction with tremelimumab (also known as ticilimumab or CP-675,206). In some instances, an immune checkpoint inhibitor (e.g., a PD-1 axis binding antagonist (e.g., an anti-PD-L1 antibody such as atezolizumab)) may be administered in conjunction with an antagonist directed against B7-H3 (also known as CD276), e.g., a blocking antibody. In some instances, an immune checkpoint inhibitor (e.g., a PD-1 axis binding antagonist (e.g., an anti-PD-L1 antibody such as atezolizumab)) may be administered in conjunction with MGA271. In some instances, an immune checkpoint inhibitor (e.g., a PD-1 axis binding antagonist (e.g., an anti-PD-L1 antibody such as atezolizumab)) may be administered in conjunction with an antagonist directed against a TGF-beta, e.g., metelimumab (also known as CAT-192), fresolimumab (also known as GC1008), or LY2157299.

In some instances, an immune checkpoint inhibitor (e.g., a PD-1 axis binding antagonist (e.g., an anti-PD-L1 antibody such as atezolizumab)) may be administered in conjunction with a treatment comprising adoptive transfer of a T-cell (e.g., a cytotoxic T-cell or CTL) expressing a chimeric antigen receptor (CAR). In some instances, an immune checkpoint inhibitor (e.g., a PD-1 axis binding antagonist (e.g., an anti-PD-L1 antibody such as atezolizumab)) may be administered in conjunction with a treatment comprising adoptive transfer of a T-cell comprising a dominant-negative TGF beta receptor, e.g., a dominant-negative TGF beta type II receptor. In some instances, an immune checkpoint inhibitor (e.g., a PD-1 axis binding antagonist (e.g., an anti-PD-L1 antibody such as atezolizumab)) may be administered in conjunction with a treatment comprising a HERCREEM protocol (see, e.g., ClinicalTrials.gov Identifier NCT00889954).

In some instances, an immune checkpoint inhibitor (e.g., a PD-1 axis binding antagonist (e.g., an anti-PD-L1 antibody such as atezolizumab)) may be administered in conjunction with an agonist directed against CD137 (also known as TNFRSF9, 4-1BB, or ILA), e.g., an activating antibody. In some instances, an immune checkpoint inhibitor (e.g., a PD-1 axis binding antagonist (e.g., an anti-PD-L1 antibody such as atezolizumab)) may be administered in conjunction with urelumab (also known as BMS-663513). In some instances, an immune checkpoint inhibitor (e.g., a PD-1 axis binding antagonist (e.g., an anti-PD-L1 antibody such as atezolizumab)) may be administered in conjunction with an agonist directed against CD40, e.g., an activating antibody. In some instances, an immune checkpoint inhibitor (e.g., a PD-1 axis binding antagonist (e.g., an anti-PD-L1 antibody such as atezolizumab)) may be administered in conjunction with CP-870893. In some instances, an immune checkpoint inhibitor (e.g., a PD-1 axis binding antagonist (e.g., an anti-PD-L1 antibody such as atezolizumab)) may be administered in conjunction with an agonist directed against OX40 (also known as CD134), e.g., an activating antibody. In some instances, an immune checkpoint inhibitor (e.g., a PD-1 axis binding antagonist (e.g., an anti-PD-L1 antibody such as atezolizumab)) may be administered in conjunction with an anti-OX40 antibody (e.g., AgonOX). In some instances, an immune checkpoint inhibitor (e.g., a PD-1 axis binding antagonist (e.g., an anti-PD-L1 antibody such as atezolizumab)) may be administered in conjunction with an agonist directed against CD27, e.g., an activating antibody. In some instances, an immune checkpoint inhibitor (e.g., a PD-1 axis binding antagonist (e.g., an anti-PD-L1 antibody such as atezolizumab)) may be administered in conjunction with CDX-1127. In some instances, an immune checkpoint inhibitor (e.g., a PD-1 axis binding antagonist (e.g., an anti-PD-L1 antibody such as atezolizumab)) may be administered in conjunction with an antagonist directed against indoleamine-2,3-dioxygenase (IDO). In some instances, the IDO antagonist is 1-methyl-D-tryptophan (also known as 1-D-MT).

In some instances, an immune checkpoint inhibitor (e.g., a PD-1 axis binding antagonist (e.g., an anti-PD-L1 antibody such as atezolizumab)) may be administered in conjunction with an antibody-drug conjugate. In some instances, the antibody-drug conjugate comprises mertansine or monomethyl auristatin E (MMAE). In some instances, an immune checkpoint inhibitor (e.g., a PD-1 axis binding antagonist (e.g., an anti-PD-L1 antibody such as atezolizumab)) may be administered in conjunction with an anti-NaPi2b antibody-MMAE conjugate (also known as DNIB0600A or RG7599). In some instances, an immune checkpoint inhibitor (e.g., a PD-1 axis binding antagonist (e.g., an anti-PD-L1 antibody such as atezolizumab)) may be administered in conjunction with trastuzumab emtansine (also known as T-DM1, ado-trastuzumab emtansine, or KADCYLA®, Genentech). In some instances, an immune checkpoint inhibitor (e.g., a PD-1 axis binding antagonist (e.g., an anti-PD-L1 antibody such as atezolizumab)) may be administered in conjunction with DMUC5754A. In some instances, an immune checkpoint inhibitor (e.g., a PD-1 axis binding antagonist (e.g., an anti-PD-L1 antibody such as atezolizumab)) may be administered in conjunction with an antibody-drug conjugate targeting the endothelin B receptor (EDNBR), e.g., an antibody directed against EDNBR conjugated with MMAE.

In some instances, an immune checkpoint inhibitor (e.g., a PD-1 axis binding antagonist (e.g., an anti-PD-L1 antibody such as atezolizumab)) may be administered in conjunction with an anti-angiogenesis agent. In some instances, an immune checkpoint inhibitor (e.g., a PD-1 axis binding antagonist (e.g., an anti-PD-L1 antibody such as atezolizumab)) may be administered in conjunction with an antibody directed against a VEGF, e.g., VEGF-A. In some instances, an immune checkpoint inhibitor (e.g., a PD-1 axis binding antagonist (e.g., an anti-PD-L1 antibody such as atezolizumab)) may be administered in conjunction with bevacizumab (also known as AVASTIN®, Genentech). In some instances, an immune checkpoint inhibitor (e.g., a PD-1 axis binding antagonist (e.g., an anti-PD-L1 antibody such as atezolizumab)) may be administered in conjunction with an antibody directed against angiopoietin 2 (also known as Ang2). In some instances, an immune checkpoint inhibitor (e.g., a PD-1 axis binding antagonist (e.g., an anti-PD-L1 antibody such as atezolizumab)) may be administered in conjunction with MEDI3617.

In some instances, an immune checkpoint inhibitor (e.g., a PD-1 axis binding antagonist (e.g., an anti-PD-L1 antibody such as atezolizumab)) may be administered in conjunction with an antineoplastic agent. In some instances, an immune checkpoint inhibitor (e.g., a PD-1 axis binding antagonist (e.g., an anti-PD-L1 antibody such as atezolizumab)) may be administered in conjunction with an agent targeting CSF-1R (also known as M-CSFR or CD115). In some instances, an immune checkpoint inhibitor (e.g., a PD-1 axis binding antagonist (e.g., an anti-PD-L1 antibody such as atezolizumab)) may be administered in conjunction with anti-CSF-1R (also known as IMC-CS4). In some instances, an immune checkpoint inhibitor (e.g., a PD-1 axis binding antagonist (e.g., an anti-PD-L1 antibody such as atezolizumab)) may be administered in conjunction with an interferon, for example interferon alpha or interferon gamma. In some instances, an immune checkpoint inhibitor (e.g., a PD-1 axis binding antagonist (e.g., an anti-PD-L1 antibody such as atezolizumab)) may be administered in conjunction with Roferon-A (also known as recombinant Interferon alpha-2a). In some instances, an immune checkpoint inhibitor (e.g., a PD-1 axis binding antagonist (e.g., an anti-PD-L1 antibody such as atezolizumab)) may be administered in conjunction with GM-CSF (also known as recombinant human granulocyte macrophage colony stimulating factor, rhu GM-CSF, sargramostim, or LEUKINE®). In some instances, an immune checkpoint inhibitor (e.g., a PD-1 axis binding antagonist (e.g., an anti-PD-L1 antibody such as atezolizumab)) may be administered in conjunction with IL-2 (also known as aldesleukin or PROLEUKIN®). In some instances, an immune checkpoint inhibitor (e.g., a PD-1 axis binding antagonist (e.g., an anti-PD-L1 antibody such as atezolizumab)) may be administered in conjunction with IL-12. In some instances, an immune checkpoint inhibitor (e.g., a PD-1 axis binding antagonist (e.g., an anti-PD-L1 antibody such as atezolizumab)) may be administered in conjunction with an antibody targeting CD20. In some instances, the antibody targeting CD20 is obinutuzumab (also known as GA101 or GAZYVA®) or rituximab. In some instances, an immune checkpoint inhibitor (e.g., a PD-1 axis binding antagonist (e.g., an anti-PD-L1 antibody such as atezolizumab)) may be administered in conjunction with an antibody targeting GITR. In some instances, the antibody targeting GITR is TRX518.

In some instances, an immune checkpoint inhibitor (e.g., a PD-1 axis binding antagonist (e.g., an anti-PD-L1 antibody such as atezolizumab)) may be administered in conjunction with a cancer vaccine. In some instances, the cancer vaccine is a peptide cancer vaccine, which in some instances is a personalized peptide vaccine. In some instances, the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, an immune checkpoint inhibitor (e.g., a PD-1 axis binding antagonist (e.g., an anti-PD-L1 antibody such as atezolizumab)) may be administered in conjunction with an adjuvant. In some instances, an immune checkpoint inhibitor (e.g., a PD-1 axis binding antagonist (e.g., an anti-PD-L1 antibody such as atezolizumab)) may be administered in conjunction with a treatment comprising a TLR agonist, e.g., Poly-ICLC (also known as HILTONOL®), LPS, MPL, or CpG ODN. In some instances, an immune checkpoint inhibitor (e.g., a PD-1 axis binding antagonist (e.g., an anti-PD-L1 antibody such as atezolizumab)) may be administered in conjunction with tumor necrosis factor (TNF) alpha. In some instances, an immune checkpoint inhibitor (e.g., a PD-1 axis binding antagonist (e.g., an anti-PD-L1 antibody such as atezolizumab)) may be administered in conjunction with IL-1. In some instances, an immune checkpoint inhibitor (e.g., a PD-1 axis binding antagonist (e.g., an anti-PD-L1 antibody such as atezolizumab)) may be administered in conjunction with HMGB1. In some instances, an immune checkpoint inhibitor (e.g., a PD-1 axis binding antagonist (e.g., an anti-PD-L1 antibody such as atezolizumab)) may be administered in conjunction with an IL-10 antagonist. In some instances, an immune checkpoint inhibitor (e.g., a PD-1 axis binding antagonist (e.g., an anti-PD-L1 antibody such as atezolizumab)) may be administered in conjunction with an IL-4 antagonist. In some instances, an immune checkpoint inhibitor (e.g., a PD-1 axis binding antagonist (e.g., an anti-PD-L1 antibody such as atezolizumab)) may be administered in conjunction with an IL-13 antagonist. In some instances, an immune checkpoint inhibitor (e.g., a PD-1 axis binding antagonist (e.g., an anti-PD-L1 antibody such as atezolizumab)) may be administered in conjunction with an HVEM antagonist. In some instances, an immune checkpoint inhibitor (e.g., a PD-1 axis binding antagonist (e.g., an anti-PD-L1 antibody such as atezolizumab)) may be administered in conjunction with an ICOS agonist, e.g., by administration of ICOS-L, or an agonistic antibody directed against ICOS. In some instances, an immune checkpoint inhibitor (e.g., a PD-1 axis binding antagonist (e.g., an anti-PD-L1 antibody such as atezolizumab)) may be administered in conjunction with a treatment targeting CX3CL1. In some instances, an immune checkpoint inhibitor (e.g., a PD-1 axis binding antagonist (e.g., an anti-PD-L1 antibody such as atezolizumab)) may be administered in conjunction with a treatment targeting CXCL9. In some instances, an immune checkpoint inhibitor (e.g., a PD-1 axis binding antagonist (e.g., an anti-PD-L1 antibody such as atezolizumab)) may be administered in conjunction with a treatment targeting CXCL10. In some instances, an immune checkpoint inhibitor (e.g., a PD-1 axis binding antagonist (e.g., an anti-PD-L1 antibody such as atezolizumab)) may be administered in conjunction with a treatment targeting CCL5. In some instances, an immune checkpoint inhibitor (e.g., a PD-1 axis binding antagonist (e.g., an anti-PD-L1 antibody such as atezolizumab)) may be administered in conjunction with an LFA-1 or ICAM1 agonist. In some instances, an immune checkpoint inhibitor (e.g., a PD-1 axis binding antagonist (e.g., an anti-PD-L1 antibody such as atezolizumab)) may be administered in conjunction with a Selectin agonist.

In some instances, an immune checkpoint inhibitor (e.g., a PD-1 axis binding antagonist (e.g., an anti-PD-L1 antibody such as atezolizumab)) may be administered in conjunction with a targeted therapy. In some instances, an immune checkpoint inhibitor (e.g., a PD-1 axis binding antagonist (e.g., an anti-PD-L1 antibody such as atezolizumab)) may be administered in conjunction with an inhibitor of B-Raf. In some instances, an immune checkpoint inhibitor (e.g., a PD-1 axis binding antagonist (e.g., an anti-PD-L1 antibody such as atezolizumab)) may be administered in conjunction with vemurafenib (also known as ZELBORAF®). In some instances, an immune checkpoint inhibitor (e.g., a PD-1 axis binding antagonist (e.g., an anti-PD-L1 antibody such as atezolizumab)) may be administered in conjunction with dabrafenib (also known as TAFINLAR®). In some instances, an immune checkpoint inhibitor (e.g., a PD-1 axis binding antagonist (e.g., an anti-PD-L1 antibody such as atezolizumab)) may be administered in conjunction with erlotinib (also known as TARCEVA®). In some instances, an immune checkpoint inhibitor (e.g., a PD-1 axis binding antagonist (e.g., an anti-PD-L1 antibody such as atezolizumab)) may be administered in conjunction with an inhibitor of a MEK, such as MEK1 (also known as MAP2K1) or MEK2 (also known as MAP2K2). In some instances, an immune checkpoint inhibitor (e.g., a PD-1 axis binding antagonist (e.g., an anti-PD-L1 antibody such as atezolizumab)) may be administered in conjunction with cobimetinib (also known as GDC-0973 or XL-518). In some instances, an immune checkpoint inhibitor (e.g., a PD-1 axis binding antagonist (e.g., an anti-PD-L1 antibody such as atezolizumab)) may be administered in conjunction with trametinib (also known as MEKINIST®). In some instances, an immune checkpoint inhibitor (e.g., a PD-1 axis binding antagonist (e.g., an anti-PD-L1 antibody such as atezolizumab)) may be administered in conjunction with an inhibitor of K-Ras. In some instances, an immune checkpoint inhibitor (e.g., a PD-1 axis binding antagonist (e.g., an anti-PD-L1 antibody such as atezolizumab)) may be administered in conjunction with an inhibitor of c-Met. In some instances, an immune checkpoint inhibitor (e.g., a PD-1 axis binding antagonist (e.g., an anti-PD-L1 antibody such as atezolizumab)) may be administered in conjunction with onartuzumab (also known as MetMAb). In some instances, an immune checkpoint inhibitor (e.g., a PD-1 axis binding antagonist (e.g., an anti-PD-L1 antibody such as atezolizumab)) may be administered in conjunction with an inhibitor of Alk. In some instances, an immune checkpoint inhibitor (e.g., a PD-1 axis binding antagonist (e.g., an anti-PD-L1 antibody such as atezolizumab)) may be administered in conjunction with AF802 (also known as CH5424802 or alectinib). In some instances, an immune checkpoint inhibitor (e.g., a PD-1 axis binding antagonist (e.g., an anti-PD-L1 antibody such as atezolizumab)) may be administered in conjunction with an inhibitor of a phosphatidylinositol 3-kinase (PI3K). In some instances, an immune checkpoint inhibitor (e.g., a PD-1 axis binding antagonist (e.g., an anti-PD-L1 antibody such as atezolizumab)) may be administered in conjunction with BKM120. In some instances, an immune checkpoint inhibitor (e.g., a PD-1 axis binding antagonist (e.g., an anti-PD-L1 antibody such as atezolizumab)) may be administered in conjunction with idelalisib (also known as GS-1101 or CAL-101). In some instances, an immune checkpoint inhibitor (e.g., a PD-1 axis binding antagonist (e.g., an anti-PD-L1 antibody such as atezolizumab)) may be administered in conjunction with perifosine (also known as KRX-0401). In some instances, an immune checkpoint inhibitor (e.g., a PD-1 axis binding antagonist (e.g., an anti-PD-L1 antibody such as atezolizumab)) may be administered in conjunction with an inhibitor of an Akt. In some instances, an immune checkpoint inhibitor (e.g., a PD-1 axis binding antagonist (e.g., an anti-PD-L1 antibody such as atezolizumab)) may be administered in conjunction with MK2206. In some instances, an immune checkpoint inhibitor (e.g., a PD-1 axis binding antagonist (e.g., an anti-PD-L1 antibody such as atezolizumab)) may be administered in conjunction with GSK690693. In some instances, an immune checkpoint inhibitor (e.g., a PD-1 axis binding antagonist (e.g., an anti-PD-L1 antibody such as atezolizumab)) may be administered in conjunction with GDC-0941. In some instances, an immune checkpoint inhibitor (e.g., a PD-1 axis binding antagonist (e.g., an anti-PD-L1 antibody such as atezolizumab)) may be administered in conjunction with an inhibitor of mTOR. In some instances, an immune checkpoint inhibitor (e.g., a PD-1 axis binding antagonist (e.g., an anti-PD-L1 antibody such as atezolizumab)) may be administered in conjunction with sirolimus (also known as rapamycin). In some instances, an immune checkpoint inhibitor (e.g., a PD-1 axis binding antagonist (e.g., an anti-PD-L1 antibody such as atezolizumab)) may be administered in conjunction with temsirolimus (also known as CCI-779 or TORISEL®). In some instances, an immune checkpoint inhibitor (e.g., a PD-1 axis binding antagonist (e.g., an anti-PD-L1 antibody such as atezolizumab)) may be administered in conjunction with everolimus (also known as RAD001). In some instances, an immune checkpoint inhibitor (e.g., a PD-1 axis binding antagonist (e.g., an anti-PD-L1 antibody such as atezolizumab)) may be administered in conjunction with ridaforolimus (also known as AP-23573, MK-8669, or deforolimus). In some instances, an immune checkpoint inhibitor (e.g., a PD-1 axis binding antagonist (e.g., an anti-PD-L1 antibody such as atezolizumab)) may be administered in conjunction with OSI-027. In some instances, an immune checkpoint inhibitor (e.g., a PD-1 axis binding antagonist (e.g., an anti-PD-L1 antibody such as atezolizumab)) may be administered in conjunction with AZD8055. In some instances, an immune checkpoint inhibitor (e.g., a PD-1 axis binding antagonist (e.g., an anti-PD-L1 antibody such as atezolizumab)) may be administered in conjunction with INK128. In some instances, an immune checkpoint inhibitor (e.g., a PD-1 axis binding antagonist (e.g., an anti-PD-L1 antibody such as atezolizumab)) may be administered in conjunction with a dual PI3K/mTOR inhibitor. In some instances, an immune checkpoint inhibitor (e.g., a PD-1 axis binding antagonist (e.g., an anti-PD-L1 antibody such as atezolizumab)) may be administered in conjunction with XL765. In some instances, an immune checkpoint inhibitor (e.g., a PD-1 axis binding antagonist (e.g., an anti-PD-L1 antibody such as atezolizumab)) may be administered in conjunction with GDC-0980. In some instances, an immune checkpoint inhibitor (e.g., a PD-1 axis binding antagonist (e.g., an anti-PD-L1 antibody such as atezolizumab)) may be administered in conjunction with BEZ235 (also known as NVP-BEZ235). In some instances, an immune checkpoint inhibitor (e.g., a PD-1 axis binding antagonist (e.g., an anti-PD-L1 antibody such as atezolizumab)) may be administered in conjunction with BGT226. In some instances, an immune checkpoint inhibitor (e.g., a PD-1 axis binding antagonist (e.g., an anti-PD-L1 antibody such as atezolizumab)) may be administered in conjunction with GSK2126458. In some instances, an immune checkpoint inhibitor (e.g., a PD-1 axis binding antagonist (e.g., an anti-PD-L1 antibody such as atezolizumab)) may be administered in conjunction with PF-04691502. In some instances, an immune checkpoint inhibitor (e.g., a PD-1 axis binding antagonist (e.g., an anti-PD-L1 antibody such as atezolizumab)) may be administered in conjunction with PF-05212384 (also known as PKI-587).

In some aspects, atezolizumab may be administered in combination with paclitaxel, nab-paclitaxel, carboplatin, cisplatin, bevacizumab, pemetrexed, gemcitabine, cobimetinib, etoposide, or a combination thereof. In some aspects, atezolizumab may be administered in combination with paclitaxel. In some aspects, atezolizumab may be administered in combination with paclitaxel and carboplatin. In some aspects, atezolizumab may be administered in combination with bevacizumab, paclitaxel, and carboplatin. In some aspects, atezolizumab may be administered with bevacizumab. In some aspects, atezolizumab may be administered in combination with nab-paclitaxel and carboplatin. In some aspects, atezolizumab may be administered in combination with nab-paclitaxel. In some aspects, atezolizumab may be administered with carboplatin and pemetrexed. In some aspects, atezolizumab may be administered with cisplatin and pemetrexed. In some aspects, atezolizumab may be administered with cobimetinib. In some aspects, atezolizumab may be administered with carboplatin and etoposide.

### IV. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used.

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, K_{D}036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:

(a) a heavy chain variable region (VH) comprising
the amino acid sequence:
(SEQ ID NO: 9)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,
and
(b) a light chain variable region (VL) comprising
the amino acid sequence:
(SEQ ID NO: 10)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence:
(SEQ ID NO: 1)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,
and
(b) the light chain amino acid sequence:
(SEQ ID NO: 2)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (Medimmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

### V. Articles of Manufacture and Kits

In another aspect of the disclosure, an article of manufacture or kit containing materials useful for the diagnostic (e.g., predictive or prognostic assessment) and/or treatment of individuals is provided.

In some instances, such articles of manufacture or kits can be used to identify an individual having a cancer (e.g., an ovarian cancer, a fallopian tube cancer, a primary peritoneal cancer, a lung cancer, a bladder cancer, a kidney cancer, breast cancer, or a skin cancer) who has an increased likelihood of experiencing treatment-induced liver dysfunction during treatment comprising an immune checkpoint inhibitor (e.g., a PD-1 axis binding antagonist (e.g., an anti-PD-L1 antibody such as atezolizumab)). Such articles of manufacture or kits may include (a) reagents for determining the polygenic risk score (PRS) of an individual for liver damage, e.g., as described in Section IIA, and (b) instructions for using the reagents to identify an individual having a cancer (e.g., an ovarian cancer, a fallopian tube cancer, a primary peritoneal cancer, a lung cancer, a bladder cancer, a kidney cancer, breast cancer, or a skin cancer) who has an increased likelihood of experiencing treatment-induced liver dysfunction during treatment comprising an immune checkpoint inhibitor (e.g., a PD-1 axis binding antagonist (e.g., an anti-PD-L1 antibody such as atezolizumab)).

In some aspects, such articles of manufacture or kits include an immune checkpoint inhibitor (e.g., a PD-1 axis binding antagonist (e.g., an anti-PD-L1 antibody such as atezolizumab)) for treating an individual with a cancer (e.g., an ovarian cancer, a fallopian tube cancer, a primary peritoneal cancer, a lung cancer, a bladder cancer, a kidney cancer, breast cancer, or a skin cancer). In some aspects, the article of manufacture or kit includes (a) an immune checkpoint inhibitor (e.g., a PD-1 axis binding antagonist (e.g., an anti-PD-L1 antibody such as atezolizumab)) and (b) a package insert including instructions for administration of an immune checkpoint inhibitor (e.g., the PD-1 axis binding antagonist (e.g., the anti-PD-L1 antibody such as atezolizumab)) to an individual having a cancer (e.g., an ovarian cancer, a fallopian tube cancer, a primary peritoneal cancer, a lung cancer, a bladder cancer, a kidney cancer, breast cancer, or a skin cancer), wherein, prior to treatment, the polygenic risk score (PRS) of an individual for liver damage in a sample from the individual has been determined and is the same as or above a liver damage reference PRS.

Any of the articles of manufacture or kits described may include a carrier means being compartmentalized to receive in close confinement one or more container means such as vials, tubes, and the like, each of the container means comprising one of the separate elements to be used in the method. Where the article of manufacture or kit utilizes nucleic acid hybridization to detect the target nucleic acid, the kit may also have containers containing nucleotide(s) for amplification of the target nucleic acid sequence and/or a container comprising a reporter-means, such as an enzymatic, fluorescent, or radioisotope label.

In some aspects, the article of manufacture or kit includes the container described above and one or more other containers including materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above. For example, the article of manufacture or kit may further include a container including a pharmaceutically-acceptable buffer, such as bacteriostatic water for injection (BWFI), phosphate-buffered saline, Ringer's solution, and dextrose solution.

The articles of manufacture or kits described herein may have a number of aspects. In one aspect, the article of manufacture or kit includes a container, a label on said container, and a composition contained within said container, wherein the composition includes one or more polynucleotides that hybridize to a complement of a locus described herein under stringent conditions, and the label on said container indicates that the composition can be used to evaluate the presence of a gene listed herein in a sample, or of a single-nucleotide polymorphism (SNP) described herein in a sample, and wherein the kit includes instructions for using the polynucleotide(s) for evaluating the presence of the gene RNA or DNA or the presence of the SNP in a particular sample type.

For oligonucleotide-based articles of manufacture or kits, the article of manufacture or kit can include, for example: (1) an oligonucleotide, e.g., a detectably labeled oligonucleotide, which hybridizes to a nucleic acid sequence encoding a protein or (2) a pair of primers useful for amplifying a nucleic acid molecule. The article of manufacture or kit can also include, e.g., a buffering agent, a preservative, or a protein stabilizing agent. The article of manufacture or kit can further include components necessary for detecting the detectable label (e.g., an enzyme or a substrate). The article of manufacture or kit can further include components necessary for analyzing the sequence of a sample (e.g., a restriction enzyme or a buffer). The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

### VI. Examples

The following are examples of methods and compositions of the disclosure. It is understood that various other aspects may be practiced, given the general description provided above, and the examples are not intended to limit the scope of the claims.

### Example 1. Treatment with Anti-PD-L1 Therapy is Associated with Increased Risk of Hepatic Immune-Related Adverse Events (irAEs)

i. Introduction

PD-1 axis binding antagonists (e.g., atezolizumab) have made significant advances in the treatment of cancer. Considerable progress has been made in identifying the immune mechanisms that are responsible for the therapeutic benefit observed. These include the enhancement of T-cell priming and activation at the level of dendritic cells and the re-invigoration of “exhausted” intra-tumoral T-cells. However, PD-1 axis binding antagonists (e.g., atezolizumab) act systemically and patients can develop immune toxicities and rheumatic complications, termed immune-related adverse events (irAEs). Activation of the immune system due to diminished activity of the PD-1 checkpoint is hypothesized to contribute to autoimmunity that increases risk for irAEs, but the underlying risk factors and mechanisms are poorly understood. In cancer patients treated with PD-1 axis binding antagonists (e.g., atezolizumab), there exists considerable inter-individual variation in tumor response and immune toxicity. Understanding an individual and their tumor's immunological status, or cancer-immune set point, can explain this variation and identify therapeutic approaches to improve not only efficacy, but also safety. This approach requires consideration of factors both intrinsic and extrinsic to a tumor, including genetic variation that affects the immune system.

Hepatic irAEs occur in 1-20% of patients treated with checkpoint inhibitors, likely depending on number, type, and dose of the treatment. Management typically involves stepwise escalation, withholding or ceasing of immunotherapy treatment, and/or corticosteroid or other immunosuppressive treatment.

ii. Hepatic irAE are Common in Cancer Patients Treated with PD-1 Axis Binding Antagonists

Activation of systemic immune responses using PD-1 axis binding antagonists (e.g., atezolizumab) is an important approach to cancer therapy. Yet, the extent of benefit relative to risk of immune related adverse events (irAEs) varies widely between patients. To characterize their prevalence, hepatic irAEs were aggregated across the safety evaluable populations of 15 atezolizumab clinical trials testing atezolizumab alone or in combinations with chemotherapies, bevacizumab, cobimetinib, and spanning 7 cancer indications: urothelial carcinoma (IMvigor211, IMvigor010), squamous and non-squamous non-small cell lung cancer (IMpower010, IMpower110, IMpower130, IMpower131, IMpower132, IMpower150), small cell lung cancer (IMpower133), metastatic renal cell carcinoma (IMmotion151), triple-negative breast cancer (TNBC) (IMpassion130, IMpassion131, IMpassion031), skin melanoma (IMspire170), and ovarian, fallopian tube, or primary peritoneal cancer (IMagyn050) (Tables 1 and 2). Protocols for each trial are provided in the original study publications (Table 1).

TABLE 1
Atezolizumab trials

| Trial | Indication | Citation | NCT Number |
|---|---|---|---|
| IMvigor211 | Locally Advanced or | Powles et al., Lancet, 391: 748-757, | NCT02302807 |
| | Metastatic Urothelial | 2018; van der Heijden et al., Eur. | |
| | Bladder Cancer | Urol., 80(1): 7-11, 2021. | |
| IMpassion131 | Previously Untreated | Miles et al., Ann. Oncol., 32(8): 994- | NCT03125902 |
| | Locally Advanced or | 1004, 2021. | |
| | Metastatic TNBC | | |
| IMpower110 | Stage IV Non-Squamous | Herbst et al., N. Engl. J. Med., | NCT02409342 |
| | or Squamous NSCLC | 383(14): 1328-1339, 2020. | |
| IMpower150 | Stage IV Non-Squamous | Socinski et al., N. Engl. J. Med., 378: | NCT02366143 |
| | NSCLC | 2288-2301, 2018. | |
| IMpower131 | Stage IV Squamous | Jotte et al., J. Thorac. Oncol., 15(8): | NCT02367794 |
| | NSCLC | 1351-1360, 2020. | |
| IMpower132 | Chemotherapy-Naive | Lu et al., Cancer Med, 12(3): 2666- | NCT02657434 |
| | Stage IV Non-Squamous | 2676, 2023; Ton et al., Clin. Cancer | |
| | NSCLC | Res, 28(13): 2844-2853, 2022. | |
| IMpower130 | Stage IV Non-Squamous | West et al., Lancet Oncol., 20: 924- | NCT02367781 |
| | NSCLC | 937, 2019. | |
| IMpower010 | Stage IB-Stage IIIA | Felip et al., Lancet, 398(10308): 1344- | NCT02486718 |
| | NSCLC | 1357, 2021. | |
| IMagyn050 | Newly-Diagnosed Stage III | Moore et al., J. Clin. Oncol., | NCT03038100 |
| | or Stage IV Ovarian, | 39(17): 1842-1855, 2021; Moore et al., | |
| | Fallopian Tube, or Primary | Int. J. Gynecol. Cancer, 2019. | |
| | Peritoneal Cancer | | |
| IMmotion151 | Untreated Advanced RCC | Rini et al., Lancet, 393: 2404-2415, | NCT02420821 |
| | | 2019. | |
| IMspire170 | Untreated Advanced | de Azevedo et al., Pigment Cell | NCT03273153 |
| | BRAFv600 Wild-Type | Melanoma Res., 34(5): 973-977, 2021. | |
| | Melanoma | | |
| IMpower133 | Untreated Extensive-Stage | Horn et al, N. Engl. J. Med., 379: | NCT02763579 |
| | SCLC | 2220-2229, 2018. | |
| IMpassion130 | Previously Untreated | Schmid et al., N. Engl. J. Med., 379: | NCT02425891 |
| | Metastatic TNBC | 2108-2121, 2018; Schmid et al., | |
| | | Lancet Oncol., 21: 44-59, 2020. | |
| IMpassion031 | Early Stage TNBC | Mittendorf et al., Lancet, | NCT03197935 |
| | | 396(10257): 1090-1100, 2020. | |
| IMvigor010 | High-Risk Muscle-Invasive | Bellmunt et al., Lancet Oncol., | NCT02450331 |
| | Urothelial Carcinoma | 22(4): 525-537, 2021. | |

NSCLC: non-small cell lung cancer; SCLC: small cell lung cancer; RCC: renal cell carcinoma; TNBC = triple negative breast cancer.

TABLE 2
Number of patients in the safety evaluable population

| | Arm | | | |
|---|---|---|---|---|
| Trial | Abbreviation | N (Safety) | N (QC) | N(Hepatitis) |
| IMagyn050 | A | 642 | 346 | 57 |
| IMagyn050 | Pl | 644 | 354 | 38 |
| IMmotion151 | AB | 451 | 215 | 33 |
| IMmotion151 | S | 446 | 197 | 36 |
| IMpassion031 | ANp | 164 | 17 | 3 |
| IMpassion031 | PlNp | 167 | 24 | 3 |
| IMpassion130 | A | 460 | 158 | 25 |
| IMpassion130 | Pl | 430 | 139 | 21 |
| IMpassion131 | A | 432 | 95 | 18 |
| IMpassion131 | Pl | 217 | 45 | 5 |
| IMpower010 | Sc | 495 | 177 | 9 |
| IMpower010 | A | 495 | 186 | 32 |
| IMpower110 | CciPmg | 263 | 82 | 8 |
| IMpower110 | A | 286 | 105 | 14 |
| IMpower130 | CNp | 232 | 103 | 9 |
| IMpower130 | ACNp | 473 | 204 | 22 |
| IMpower131 | CNp | 334 | 159 | 13 |
| IMpower131 | ACNp | 334 | 160 | 28 |
| IMpower131 | ACP | 332 | 165 | 29 |
| IMpower132 | ACPm | 180 | 82 | 17 |
| IMpower132 | ACiPm | 192 | 43 | 7 |
| IMpower132 | CPm | 169 | 72 | 13 |
| IMpower132 | CiPm | 186 | 35 | 1 |
| IMpower133 | ACE | 250 | 77 | 5 |
| IMpower133 | PlCE | 244 | 71 | 2 |
| IMpower150 | CPB | 394 | 187 | 7 |
| IMpower150 | ACP | 400 | 228 | 26 |
| IMpower150 | ACPB | 393 | 209 | 29 |
| IMspire170 | ACo | 220 | 142 | 44 |
| IMvigor010 | A | 390 | 199 | 20 |
| IMvigor010 | O | 403 | 198 | 0 |
| IMvigor211 | Ch | 463 | 214 | 16 |
| IMvigor211 | A | 468 | 229 | 23 |

Number of patients (Total) that met genotype and population QC filters (QC), and had hepatitis (Hepatitis) separated by trial and arm. Abbreviations: A = atezolizumab; B = bevacizumab; C = carboplatin; Cci = carboplatin or cisplatin; Ch = chemotherapy; Ci = cisplatin; Co = cobimetinib; E = etoposide; G = gemcitabine; Np = Nab-paclitaxel; P = paclitaxel; Pl = Placebo; Pm = pemetrexed; Pmg = pemetrexed or gemcitabine; S = sunitinib; Sc = supportive care;

iii. Hepatic irAEs Analyzed

irAEs were defined on the basis of an adverse events of special interest (AESI) strategy uniformly applied across studies. The original study protocols cited in Table 1 provide details on this methodology.

iv. Results

A total of 11,649 patients were in the safety evaluable population in these trials (Table 2), of which N=6,562 received atezolizumab in combination or as monotherapy. Treatment with atezolizumab as a monotherapy or in combination was associated with increased risk of hepatic irAE as compared to patients not receiving atezolizumab (Fisher's exact test, P=1.58e-18, Odds Ratio (OR)=1.6).

### Example 2. Polygenic Risk Scores Related to Liver Damage are Associated with Hepatitis irAE Risk in Individuals Treated with the Anti-PD-L1 Antibody Atezolizumab

The levels of biomarkers commonly used to assess liver health, such as serum alanine aminotransferase (ALT), aspartate aminotransferase (AST), gamma-glutamyl transferase (GGT), and alkaline phosphatase (ALP), are in part determined by common genetic variation (Sinnott-Armstrong et al., Nature Genetics, 53:185-194, 2021; Privé et al., Am. J. Hum. Genet., 109:12-23, 2022). In this Example, it was investigated whether polygenic risk scores (PRS), i.e., aggregates of genetic variants that affect ALT, AST, GGT, and ALP levels, also contribute to the risk of hepatic irAEs during atezolizumab treatment. 16 PRSs for ALT (PGS000668, PGS002158, PGS001940, PGS000816), AST (PGS000673, PGS002159, PGS001941), GGT (PGS001964, PGS002182, PGS000683, PGS000817), ALP (PGS000815, PGS000670, PGS001939, PGS002157), and cirrhosis of liver (PGS000726) were downloaded from the PGS catalog (Lambert et al., Nature Genetics, 53:420-425, 2021), and applied to 4,917 patients of European ancestry, of which 2,860 were treated with atezolizumab.

i. Whole Genome Sequencing and Sample/Variant QC of Atezolizumab Trial Cohort Genomic DNA was extracted from blood samples using the DNA Blood400 kit (C_{HEMAGIC}™) and eluted in 50 μL Elution Buffer (EB, Qiagen). DNA was sheared using a LE220 Focused-ultrasonicator (COVARIS®) and sequencing libraries were prepared using the TRUSEQ® DNA Nano HT kit (Illumina). 150 bp paired-end whole-genome sequencing (WGS) data was generated to an average read depth of 30× using the HISEQ® platform.

Reads were aligned using the functionally equivalent BAM (FEB) pipeline. Samples were jointly genotyped using the SENTIEON® genome analysis toolkit (GATK). Only variants flagged as PASS and genotype calls with GQ>20 were used. Multi-allelic sites were handled by keeping only calls for the two most common alleles; all other calls were set to missing. Samples were removed if they had a high within-sample missing rate of >0.1. Samples were then merged with 1000G samples, and LD pruned. ADMIXTURE v1.23 was used to estimate ancestry in the 5 major populations using supervised mode. Samples with >0.7 European (EUR) ancestry were extracted and analyzed for heterozygosity outliers by estimating the per sample F inbreeding coefficient. EUR samples with an F statistic more than 6 standard deviations from the mean were removed. The final PCA was then performed to compute 10 eigenvectors that were subsequently used to account for any remaining population stratification.

Using this final EUR cohort with missing rate, heterozygosity, relatedness, and PCA outlier samples removed, variant level QC was performed. Variants with genotype call missing rate of >0.1 were removed. Variants were also analyzed for violation of Hardy Weinberg equilibrium at p<5×10⁻¹⁰, and any variants with MAF<0.001 were removed.

ii. Construction and Computation of Polygenic Risk Scores

Individual scores were computed for each of the 16 PRS models using the plink2-score method with mean imputation enabled using processed genotype data. The scores were calculated based on bi-allelic variants with unique Reference SNP cluster IDs from chromosome 1-22 and X, which were included in each PRS model. The average score per variant was determined for each individual and then normalized scores across all individuals.

iii. PRS and irAE Meta-Analysis Across Trial Arms

A mixed effect Cox model was used allowing for a different baseline hazard per trial arm and controlling for 5 genotype principal components using coxme package in R: Surv(Hepatitis.irAE.time, Hepatitis.irAE.occured)˜PRS+(PRS|trial.arm)+PC1+PC2+PC3+PC4+PC5+ strata(trial.arm). The analysis included trial arms where at least 5 individuals developed hepatitis for reliable statistical analysis. The meta-analysis p-values for 16 PRSs were corrected for multiple tests using the Benjamini-Hochberg method.

iv. Correlation Between Liver Damage-Related PRS and Risk of Drug-Induced Hepatitis

16 PRS for phenotypes related to liver damage were tested for association with atezolizumab-induced hepatitis. Out of these, 4 PRSs for ALT and 3 PRSs for AST were found to be significantly associated (FDR:0.05) with hepatitis irAE risk (Table 3). No significant associations were found across the control arms of the studies (Table 4).

v. Risk of Hepatitis Gradually Increases with Liver-Damage PRS in Patients Treated with Atezolizumab

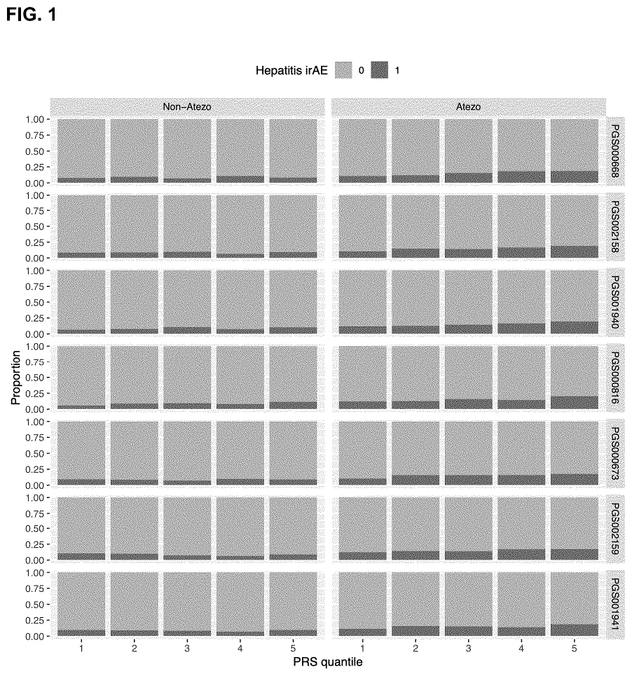

Individuals were organized into five quantiles based on their scores for each of the seven PRS (four ALT and three AST) that showed a significant association with hepatitis irAE risk. Quantile 1 represents individuals with the lowest polygenic risk scores (PRSs), while quantile 5 represents those with the highest scores. To visualize the relationship, a plot (FIG. 1) was created showing the proportion of patients with hepatitis, categorized by whether they received treatment with atezolizumab or not. Among the patients treated with atezolizumab, those in the higher quantiles of each PRS generally exhibited a higher risk of developing hepatitis.

TABLE 3
Associations between hepatitis irAE risk and PRSs in atezolizumab-treated patients

| | | | Hazard | Std. | | |
|---|---|---|---|---|---|---|
| PGS ID | Trait | Coefficient | Ratio | Error | P-value | FDR |
| PGS000668 | serum alanine aminotransferase | 0.2456 | 1.2785 | 0.0488 | 4.73E−07 | 7.57E−06 |
| | measurement | | | | | |
| PGS002158 | serum alanine aminotransferase | 0.2389 | 1.2699 | 0.0515 | 3.53E−06 | 2.82E−05 |
| | measurement | | | | | |
| PGS001940 | serum alanine aminotransferase | 0.2337 | 1.2633 | 0.0528 | 9.73E−06 | 5.19E−05 |
| | measurement | | | | | |
| PGS000816 | serum alanine aminotransferase | 0.2186 | 1.2444 | 0.0519 | 2.56E−05 | 1.03E−04 |
| | measurement | | | | | |
| PGS000673 | aspartate aminotransferase | 0.1863 | 1.2048 | 0.0485 | 1.22E−04 | 3.91E−04 |
| | measurement | | | | | |
| PGS002159 | aspartate aminotransferase | 0.1847 | 1.2028 | 0.0493 | 1.82E−04 | 4.85E−04 |
| | measurement | | | | | |
| PGS001941 | aspartate aminotransferase | 0.1643 | 1.1786 | 0.0490 | 8.07E−04 | 1.84E−03 |
| | measurement | | | | | |
| PGS001964 | serum gamma-glutamyl transferase | 0.1307 | 1.1396 | 0.0591 | 2.69E−02 | 5.38E−02 |
| | measurement | | | | | |
| PGS002182 | serum gamma-glutamyl transferase | 0.1278 | 1.1363 | 0.0603 | 3.41E−02 | 5.63E−02 |
| | measurement | | | | | |
| PGS000726 | cirrhosis of liver | 0.1023 | 1.1077 | 0.0486 | 3.52E−02 | 5.63E−02 |
| PGS000683 | serum gamma-glutamyl transferase | 0.1179 | 1.1251 | 0.0603 | 5.06E−02 | 7.35E−02 |
| | measurement | | | | | |
| PGS000815 | alkaline phosphatase measurement | 0.0849 | 1.0886 | 0.0490 | 8.32E−02 | 1.11E−01 |
| PGS000670 | alkaline phosphatase measurement | 0.0743 | 1.0771 | 0.0483 | 1.24E−01 | 1.52E−01 |
| PGS000817 | serum gamma-glutamyl transferase | 0.0483 | 1.0495 | 0.0537 | 3.68E−01 | 4.21E−01 |
| | measurement | | | | | |
| PGS001939 | alkaline phosphatase measurement | 0.0321 | 1.0326 | 0.0483 | 5.06E−01 | 5.40E−01 |
| PGS002157 | alkaline phosphatase measurement | 0.0001 | 1.0001 | 0.0485 | 9.99E−01 | 9.99E−01 |

TABLE 4
Associations between hepatitis irAE risk and PRSs in control patients

| | | | Hazard | Std. | | |
|---|---|---|---|---|---|---|
| PGS ID | Trait | Coefficient | Ratio | Error | P-value | FDR |
| PGS000816 | serum alanine aminotransferase | 0.1808 | 1.1981 | 0.0805 | 2.47E−02 | 2.72E−01 |
| | measurement | | | | | |
| PGS001940 | serum alanine aminotransferase | 0.1616 | 1.1753 | 0.0821 | 4.91E−02 | 2.72E−01 |
| | measurement | | | | | |
| PGS001964 | serum gamma-glutamyl transferase | 0.1496 | 1.1614 | 0.0784 | 5.65E−02 | 2.72E−01 |
| | measurement | | | | | |
| PGS000683 | serum gamma-glutamyl transferase | 0.1452 | 1.1562 | 0.0795 | 6.79E−02 | 2.72E−01 |
| | measurement | | | | | |
| PGS002182 | serum gamma-glutamyl transferase | 0.1271 | 1.1356 | 0.0778 | 1.02E−01 | 3.26E−01 |
| | measurement | | | | | |
| PGS002158 | serum alanine aminotransferase | 0.0852 | 1.0889 | 0.0803 | 2.89E−01 | 5.52E−01 |
| | measurement | | | | | |
| PGS002157 | alkaline phosphatase measurement | 0.0818 | 1.0852 | 0.0782 | 2.96E−01 | 5.52E−01 |
| PGS001939 | alkaline phosphatase measurement | 0.0823 | 1.0858 | 0.0826 | 3.19E−01 | 5.52E−01 |
| PGS000817 | serum gamma-glutamyl transferase | 0.0774 | 1.0805 | 0.0779 | 3.21E−01 | 5.52E−01 |
| | measurement | | | | | |
| PGS000670 | alkaline phosphatase measurement | 0.0739 | 1.0767 | 0.0783 | 3.45E−01 | 5.52E−01 |
| PGS000668 | serum alanine aminotransferase | 0.0631 | 1.0651 | 0.0777 | 4.17E−01 | 5.94E−01 |
| | measurement | | | | | |
| PGS000673 | aspartate aminotransferase | 0.0599 | 1.0617 | 0.0786 | 4.46E−01 | 5.94E−01 |
| | measurement | | | | | |
| PGS001941 | aspartate aminotransferase | 0.0501 | 1.0513 | 0.0923 | 5.88E−01 | 6.94E−01 |
| | measurement | | | | | |
| PGS000726 | cirrhosis of liver | 0.0371 | 1.0378 | 0.0722 | 6.08E−01 | 6.94E−01 |
| PGS000815 | alkaline phosphatase measurement | −0.0243 | 0.9760 | 0.0866 | 7.79E−01 | 8.31E−01 |
| PGS002159 | aspartate aminotransferase | −0.0140 | 0.9861 | 0.1014 | 8.90E−01 | 8.90E−01 |
| | measurement | | | | | |

What is claimed is:

1. A method of identifying a human individual having a cancer who has an increased likelihood of experiencing treatment-induced liver dysfunction during treatment comprising atezolizumab, the method comprising determining a polygenic risk score (PRS) for liver damage from a sample from the individual, wherein a PRS for liver damage that is above a liver damage reference PRS identifies the individual as one who may have an increased likelihood of experiencing treatment-induced liver dysfunction during treatment comprising atezolizumab.

2. A method of treating a human individual having a cancer, the method comprising:
(a) determining a PRS for liver damage from a sample from the individual, wherein the PRS for liver damage is above a liver damage reference PRS;
(b) administering an effective amount of atezolizumab to the individual; and
(c) monitoring the individual for symptoms of treatment-induced liver dysfunction.

3. The method of claim 1 or 2, wherein the liver damage reference PRS is a pre-assigned PRS.

4. The method of claim 1 or 2, wherein the liver damage reference PRS is the first, second, third, fourth, or fifth quantile PRS for liver damage in a reference population.

5. The method of claim 4, wherein the reference population is a population of individuals having the cancer.

6. The method of any one of claims 1-5, wherein (a) the PRS for liver damage of the sample from the individual or (b) the PRS for liver damage of a sample from an individual in the reference population is calculated using the equation:
$$\hat{S} = {\sum }_{i = 1}^{M} {β}_{i} \cdot {G}_{i}$$
wherein:
(i) Ŝ is the PRS for liver damage;
(ii) M is the number of risk alleles selected from independent genetic signals in a genome-wide association study (GWAS) for liver damage;
(iii) i represents the index of a given SNP;
(iv) βᵢ is the log odds ratio or conditionally independent odds ratio of the ith SNP; and
(v) Gᵢ={0,1,2} is the number of copies of the SNP in the sample from the individual.

7. The method of claim 6, wherein the risk alleles are identified in the sample by whole-genome sequencing.

8. The method of claim 6, wherein the risk alleles are the risk alleles associated with a polygenic score (PGS) having a PGS catalog identification (ID) of PGS000668, PGS002158, PGS001940, PGS000816, PGS002159, PGS000673, or PGS001941.

9. The method of any one of claims 1-8, wherein the PRS for liver damage is a PRS for serum alanine aminotransferase (ALT) or aspartate aminotransferase (AST).

10. The method of any one of claims 1-9, wherein the sample is a whole blood sample, a plasma sample, a serum sample, or a combination thereof.

11. The method of any one of claims 1-10, wherein the sample is an archival sample, a fresh sample, or a frozen sample.

12. The method of any one of claims 2-11, further comprising administering to the individual one or more additional therapeutic agents.

13. The method of claim 12, wherein the one or more additional therapeutic agents comprise paclitaxel, nab-paclitaxel, carboplatin, cisplatin, bevacizumab, pemetrexed, gemcitabine, cobimetinib, etoposide, or a combination thereof.

14. The method of any one of claims 1-13, wherein the individual has not been previously treated for the cancer.

15. The method of any one of claims 1-14, wherein the individual has not been previously administered atezolizumab.

16. The method of any one of claims 1-15, wherein the individual is of European ancestry.

17. The method of any one of claims 1-16, wherein the cancer is an ovarian cancer, a fallopian tube cancer, a primary peritoneal cancer, a lung cancer, a bladder cancer, a kidney cancer, breast cancer, or a skin cancer.

18. The method of claim 17, wherein:
(a) the ovarian cancer is a Stage III or IV ovarian cancer;
(b) the fallopian tube cancer is a Stage III or IV fallopian tube cancer;
(c) the primary peritoneal cancer is a Stage III or IV primary peritoneal cancer;
(d) the lung cancer is a small cell lung cancer (SCLC) or a non-small cell lung cancer (NSCLC);
(e) the bladder cancer is a urothelial carcinoma (UC) or a urothelial bladder cancer (UBC);
(f) the kidney cancer is a renal cell carcinoma (RCC);
(g) the breast cancer is a triple negative breast cancer (TNBC); or
(h) the skin cancer is a melanoma.

19. The method of claim 18, wherein:
(a) the SCLC is an extensive-stage (ES) SCLC;
(b) the NSCLC is a Stage IB-Stage IIIA NSCLC, a Stage IV non-squamous NSCLC or a Stage IV squamous NSCLC;
(c) the UC is a high-risk muscle-invasive UC;
(d) the UBC is a locally advanced or metastatic UBC;
(e) the RCC is an inoperable, locally advanced, or metastatic RCC;
(f) the TNBC is an early stage TNBC, a locally advanced TNBC, or a metastatic TNBC; or
(g) the melanoma is a BRAFv600 wild-type melanoma.

20. The method of any one of claims 1-19, wherein atezolizumab is administered as an adjuvant or a neoadjuvant therapy.

21. The method of any one of claims 1-20, wherein the treatment-induced liver dysfunction is hepatitis.

22. The method of any one of claims 1-20, wherein the treatment-induced liver dysfunction is a hepatic immune-related adverse event.

23. The method of any one of claims 1-22, wherein the treatment-induced liver dysfunction is determined through clinical diagnosis or measurement of laboratory values.
